(19) United States
(12) Patent Application Publication
(10) Pub. No.: US 2009/0263362 A1
(43) Pub. Date: Oct. 22, 2009

(54) FERTILITY ENHANCEMENT USING LIPID CARRIERS AND BIOACTIVE MOLECULES

(75) Inventors: Patricia A. DeLeon, Bear, DE (US); Genevieve S. Griffiths, Wilmington, DE (US)

Correspondence Address: MCCARTER & ENGLISH, LLP WILMINGTON;BASIL S. KRIKELIS, Renaissance Centre, 405 N. King Street, 8th Floor, WILMINGTON, DE, 19801, US

(73) Assignee: University of Delaware, Newark, DE (US)

(21) Appl. No.: 12/412,810

(22) Filed: Mar. 27, 2009

Related U.S. Application Data
(63) Continuation-in-part of application No. 11/928,962, filed on Oct. 30, 2007.
(60) Provisional application No. 60/855,500, filed on Oct. 31, 2006.

(51) Int. Cl. A61K 45/00; A61K 38/16; C12N 5/06; A61P 15/08

(52) U.S. Cl. 424/93.7; 514/7; 435/375

(57) ABSTRACT

The invention relates to a composition and method for enhancing fertilization. Fertilization enhancement is achieved by effectively delivering bioactive molecules with a lipid anchor (GPI-linked proteins) to the surface of epididymal or ejaculated sperm. The process may be facilitated or promoted in the presence of Clusterin/APOJ, a well-known lipid carrier. The rate of delivery, or removal, of bioactive molecules is regulated by the concentration of lipid carrier present. The acquisition of these molecules, such as Sperm Adhesion Molecule 1 (SPAM1) can significantly impact sperm maturation and function.

### CROSS REFERENCE TO RELATED APPLICATIONS

This application is a continuation-in-part of U.S. patent application Ser. No. 11/928,962, filed Oct. 30, 2007, which claims the benefit of U.S. Provisional Patent Application Ser. No. 60/855,500 filed Oct. 31, 2006, the contents of which are herein incorporated by reference.

### RELATED FEDERALLY SPONSORED RESEARCH

The work described in this application was sponsored by the National Institutes of Health (NIH) under Contract Number ROI HD38273.

### FIELD OF THE INVENTION

The invention relates to a composition and method for enhancing fertilization. Fertilization enhancement is achieved by effectively delivering bioactive molecules with a lipid anchor (GPI-linked proteins) to the surface of epididymal or ejaculated sperm. The process may be facilitated or promoted in the presence of Clusterin/APOJ, a well-known lipid carrier. The rate of delivery, or removal, of bioactive molecules is regulated by the concentration of lipid carrier present. The acquisition of these molecules, such as Sperm Adhesion Molecule 1 (SPAM1), can significantly impact sperm maturation and function.

### BACKGROUND OF THE INVENTION

Cell-to-cell transfer of glycosyl phosphatidylinositol (GPI)-linked membrane proteins in vivo is known thus far for sperm and erythrocytes, cell types in which biosynthetic ability is absent or limited. This transfer plays a pivotal role in the remodeling of the sperm plasma membrane (PM) during their maturation in both the male and female genital tracts.

Although sperm leaving the testis are incapable of transcriptional and translational activity, their surface proteins undergo a remarkable degree of modification during epididymal maturation and capacitation in the female tract. During epididymal transit (which may vary from 3-12 days depending on the species) sperm are in an intimate association with the epididymal epithelium and its secretions and thereby exposed to variety of macromolecules that are sequentially added to their PM surface. After epididymal transit, however, sperm are not fully mature and ready to fertilize an egg. During capacitation in the female, molecules are added to sperm from the secretions of the female tract, where sperm reside for a shorter period. Some of these modifications on the sperm surface result from exchanges between soluble lipid donors or acceptors and the PM, and a variety of the proteins involved are GPI-linked. After capacitation in the female tract, sperm are fully mature and ready to fertilize an egg.

Sperm surface remodeling plays an important role in fertilization. The addition of bioactive molecules, present in both the vesicular and the membrane-free soluble fraction of epipidymal and uterine luminal fluid (ELF,ULF), on the surface of sperm furthers post-testicular maturation. This remodeling increases the likelihood of successful fertilization with an egg. Deficiencies in sperm surface remodeling, likewise, lead to a reduction in egg fertilization. The inventors have discovered a new composition and method of enhancing fertilization by promoting the remodeling of the sperm surface.

### SUMMARY OF THE INVENTION

The present invention is directed to a composition comprising a substantially purified bioactive molecule, such as GPI-linked proteins, enzymes, adhesion molecules, immune proteins and glycoproteins, and about 1-150 ng/mL of a substantially purified lipid carrier. GPI-linked proteins may be sperm adhesion molecule 1 (SPAM1), P34H, CDS9, CD55 or CDw52. The lipid carrier may be a lipid transport protein, such as Clusterin, APOJ, Clusterin/APOJ, ApoA-1, SGP2, TRPM, gp80 or SP-40. The bioactive molecule may be naturally occurring, synthetic or recombinantly derived.

The present invention is also directed to a method of enhancing fertilization comprising administering to an animal, male or female, a composition comprising a substantially purified bioactive molecule and about 1-150 ng/mL of a substantially purified lipid carrier. The composition may comprise a substantially purified bioactive molecule and a lipid carrier. The composition may comprise a bioactive molecule and a substantially purified lipid carrier. The method may be used to transfer the bioactive molecule from the composition to the surface of a sperm cell in the animal.

The present invention is also directed to an in vitro method for enhancing sperm maturation and function before or after IUI, the method comprising the steps of, isolating sperm from a male candidate and combining, in vitro, said sperm in a medium supplemented with at least one lipid carrier at a concentration of about 1-150 ng/mL and/or said GPI-linked molecule and incubating for a predetermined amount of time.

The present invention is also directed to a method for enhancing sperm maturation and function before in vitro fertilization, the method comprising, the steps of isolating sperm from a male candidate, in an in vitro environment, capacitating said sperm in a capacitation medium wherein the capacitation medium is supplemented with at least one lipid carrier at a concentration of about 1-150 ng/mL or at least one protein and incubating for a predetermined amount of time.

The present invention is also directed to a method for delivery of a GPI-linked molecule, naturally or recombinantly derived, to sperm in intrauterine insemination (IUI) or in in vitro fertilization (IVF), the method comprising the step of combining, in vitro, said sperm in a medium supplemented with said GPI-linked molecule and incubating for a predetermined amount of time.

The invention is also directed to a method for in vitro fertilization, the method comprising the steps of (a) obtaining an egg from a female candidate; (b) isolating sperm from a male candidate; (c) capacitating, in vitro, said sperm in a capacitation medium supplemented with at least one lipid carrier at a concentration of about 1-150 ng/mL and/or at least one GPI-linked protein; (d) fertilizing, in vitro, said egg with sperm to produce at least one fertilized egg; (e) culturing said fertilized egg to produce an embryo; and (f) transferring at least one embryo to the uterus of an animal.

The invention is also directed to a method for intrauterine insemination, the method comprising the steps of (a) isolating sperm from a male candidate; (b) combining, in vitro, said sperm in a medium supplemented with at least one lipid carrier at a concentration of about 1-150 ng/mL and/or at least one GPI-linked protein; and (c) thereafter, introducing said sperm into the uterine tract of an animal wherein the sperm fertilizes an egg.

The invention is also directed to a method for removing a GPI-linked protein from a cell that recombinantly expresses the GPI-linked protein, the method comprising the step of adding at least 40 ug/mL of a lipid carrier (i.e. lipoproteins), or at least 150 ng/mL of APOJ, to said cell prior to removal.

The present invention is also directed to a method for enhancing sperm maturation and function comprising, in an in vitro environment: (a) obtaining a first sample of sperm from a first male candidate; (b) treating said first sample of sperm with a solution comprising lipoproteins, wherein the solution comprises an effective amount of lipoproteins to remove at least one bioactive molecule from said first sample of sperm; (c) recovering a supernatant from the treated solution, wherein said supernatant comprises said at least one bioactive molecule and said lipoproteins; and (d) adding said recovered supernatant to a second sample of sperm, whereby said at least one bioactive molecule is transferred to the surface of a sperm.

The invention is finally directed to a method for enhancing sperm maturation and function, the method comprising, in an in vitro environment, the steps of (a) obtaining a first amount of sperm from a male candidate; (b) adding to said first amount of sperm a solution comprising lipoproteins, wherein the solution comprises at least about 40 ug/ml of lipoproteins whereby said at least about 40 ug/ml of lipoproteins removes at least one bioactive molecule (e.g. a GPI-linked protein) from said first amount of sperm; (c) recovering a supernatant from the solution, wherein said supernatant comprises said at least one bioactive molecule and said at least about 40 ug/ml of lipoproteins; and (d) adding said supernatant to a medium comprising a second amount of sperm from a second male candidate, wherein said medium comprises less than about 40 ug/ml of lipoproteins and said at least one bioactive molecule is transferred from the medium to the surface of a sperm. Alternatively, the solution may initially comprise at least about 150 ng/ml of APOJ which is subsequently diluted in the medium to less than about 150 ng/ml of APOJ.

The GPI-linked proteins may be selected from the group consisting of sperm adhesion molecule 1 (SPAM1), P34H, CD59, CDS5 or CDw52. The lipid carrier may be selected from the group consisting of Clusterin, APOJ, Clusterin/APOJ, ApoA-1, SGP2, TRPM, gp80 and SP-40.

### BRIEF DESCRIPTION OF THE DRAWINGS

The file of this patent contains at least one drawing executed in color. Copies of this patent with color drawings(s) will be provided by the Patent and Trademark Office upon request and payment of the necessary fee.

FIG. 1 is an illustration of a GPI-linked protein showing the acyl chain which anchors it in the external leaflet of the lipid bylayer. The C-terminal amino acid of the protein is linked to the inositol phospholipids anchor through a core glycan structure.

FIG. 2 is a model showing lipid exchange at the surface of sperm within the epididymis and uterus.

FIG. 3 shows an association of SPAM1 and APOJ in co-immunoprecipitation (IP) from ELF and ULF. The presence (+) of APOJ and SPAM1 Ab, control preimmune serum (PIS), and the Ab used for Western blots (WB) are indicated. In (A) the ˜67 kDa SPAM1 band is seen for ELF and ULF and is precipitated by APOJ Ab. In (B), where the proteins are unreduced, an ˜70 kDa APOJ band is precipitated by SPAM1 Ab in both ULF and ELF (arrow).

FIG. 4 shows a comparison of SPAM1 uptake. SPAM1 uptake is hindered by increasing lipoprotein concentrations.

FIG. 5 shows the effect of lipoprotein concentration in ULF on SPAM1 uptake. Low concentrations of lipoprotein supplements in ULF enhance the uptake of SPAM1 measured by flow cytometric analysis.

FIG. 6 shows the effect of lipoprotein on SPAM1 uptake for human and mouse sperm. SPAM1 is removed from mouse sperm (A) after treatment with exogenous lipoproteins. The control (grey) was PBS-treated. B shows human sperm incubated in varying concentrations of lipoproteins with solubilized human sperm PM proteins. While transfer occurred in all samples, the efficiency was highest at 16 μg/mL. In A and B, 50,000 cells were analyzed for each sample by flow cytometry.

FIG. 7 shows HASGE analysis of sperm protein. HASGE analysis of 20 μg sperm protein loaded in each lane. Lane 1 has mouse proteins. Lanes 2-7 are human samples. Lane 4 has no hyaluronidase activity. Compared to 3, Lanes 2, 5-7 have varying degrees of reduced activity.

FIG. 8 shows the soluble membrane-free fraction of ELF contains SPAM1 in complexes with varying MWs. A) Native Western blot analysis showed that low MW complexes of SPAM1 were most abundant in the 230S supernatant (monomeric SPAM1) after repeat ultracentrifugation. The pellets at 120, 150, 190, and 230,000 g had most of the SPAM1 in smears, reflecting the presence of oligomeric aggregates which were diminished in the last pellet. Lane 7 had BSA used as a control to show the specificity of anti-SPAM1 antibody. B) A model depicting a possible dynamic equilibrium between oligomeric aggregates and monomers whose hydrophobic tails are stabilized by apolipoprotein(s) (lipid carriers) in LF 230S is shown.

FIG. 9 shows SPAM1 uptake occurs only from the soluble ELF 230S monomeric fraction. Flow cytometric analysis of the acquisition of SPAM1 from 230S (A and B) and 230P oligomeric fractions (B) shows that fluorescence intensity increased (shifted to the right), when compared to the BSA control, only for 230S.

FIG. 10 shows SPAM1 transfer from 230S is diminished by cleavage of the GPI anchor. After PI-PLC or carrier treatment of ELF 230S fractions, followed by addition of manoalide for inactivation of the enzyme prior to incubation of samples with sperm, transfer was analyzed by flow cytometry. In the bimodal distribution of SPAM1 quantities fluorescence intensities of both subpopulations shifted to the left for the enzyme-treated samples, representing a decrease in delivery of SPAM1 compared to the carrier-treated sample. There was a >7-fold increase in the number of sperm from the subpopulation with lower SPAM1 quantities after PI-PLC treatment when compared to the carrier-treated sample.

FIG. 11 shows that APOJ Antibody-inhibited 230S has markedly reduced levels of SPAM1 transfer. Flow Cytometric analysis in A) demonstrates that when sperm were incubated in PBS containing APOJ Ab (1:1000) for 2 hr, the APOJ Ab could be removed with 1M KCl, unlike acetic acid (pH 3.0) treatment where the APOJ fluorescence was shifted to the right of the control (grey). Thus KCL was used to strip sperm of the rabbit polyclonal APOJ Ab prior to immunostaining of bound SPAM1 with a rabbit anti-SPAMX Ab, preventing cross-reactivity of the secondary rabbit IgG. B and C demonstrate that the addition of APOJ Ab (1:1000) to ELF and ULF 230S prior to incubation with sperm inhibited SPAM1 transfer, as detected by SPAM1 fluorescence.

FIG. 12 shows that purified recombinant human APOJ has a dose-related effect on SPAM1 transfer to the sperm surface. A) shows that SPAM1 uptake is increased when sperm are incubated in 230S ELF compared to PBS-BSA controls and that there is a direct relationship between the amount of transfer and the concentration of APOJ added to ELF 230S up to 60 ng/ml. B) At APOJ concentrations higher than 60 ng/ml sperm SPAM1 is decreased dosage-dependently. This is consistent with the formation of complexes of APOJ and SPAM1 in the liquid phase and, after saturation, sequestration of sperm SPAM1 at high APOJ concentrations.

### DETAILED DESCRIPTION OF THE INVENTION

The objective of this invention is to enhance fertilization in animals.

Another objective of this invention is to supply the sperm surface with biologically or biomedically-relevant membrane-free molecules that will enhance the sperm's functional ability.

Another objective of this invention is to enhance the ability of sperm to effect fertilization in vitro, as well as after intrauterine insemination.

As used herein, the term “substantially purified” refers to naturally occurring, synthetic or recombinant compounds that are at least 80% pure. Preferably, the compounds are at least 85% pure. More preferably, the compounds are at least 90% pure. Even more preferably, the compounds are at least 95% pure. And even more preferably, the compounds are at least 99% pure. And even still more preferably, the compounds are at least 99.9% pure.

As used herein, the term “bioactive molecule” refers to a molecule that can be present or found in epididymal and/or uterine secretion. Some examples of such molecules include SPI-linked proteins, enzymes, adhesion molecules, immune proteins, antigens and glycoproteins. These bioactive molecules may be naturally occurring, synthetic or recombinantly derived. Bioactive molecules may also be referred to as “surface remodeling,” such as “surface modeling proteins.” Bioactive molecules of the invention are preferably membrane-free and have biological and/or biomedical relevance to a sperm's functional characteristics.

As used herein, the term “GPI-linked protein” refers to proteins that can attach to the surface of the sperm by glycosy phosphatidylinositol linkage, such as SPAM1, P34H, CD52 (CDw52), CD55, CD59, and CD73.

As used herein, the term “lipid transport protein” or “lipid carrier” refers to a compound that transports bioactive molecules to and from the sperm surface, such as Clusterin, APOJ, Clusterin/APOJ, ApoA-1, SCP2, TRPM, gp80 and SP-40.

As used herein, the terms “intrauterine insemination” and “in vitro fertilization” refer to such assisted reproduction methods known in the art and include intrauterine insemination (IUI), intracervical insemination, embryo transfer and gamete intrafallopian transfer. Such methods are useful for assisting males and females who may have physiological or metabolic disorders that prevent natural conception. They may be used to enable females to bear progeny who are otherwise unable to conceive naturally. In addition to use in humans, such methods are also useful in animal breeding programs, such as for livestock breeding, and could be used as methods for the creation of transgenic animals. Bioactive molecules of this invention can be combined with sperm, an egg or an egg-sperm mixture prior to fertilization of the egg. In some species, sperm capacitate under in vitro conditions spontaneously during in vitro fertilization procedures, but normally sperm capacitate over an extended period of time both in vivo and in vitro. It is advantageous to enhance sperm activation during such procedures to increase the likelihood of successful fertilization.

As used herein, the term “capacitation” and “capacitate” refer to the specific changes a sperm undergoes in the uterine tract to develop the capacity to fertilize ova, such as protein changes on the surface or associated with the plasma membrane facilitate penetration of the sperm into the ovum. Sperm maturation occurs over a continuum, which is described as three stages. The first stage of sperm maturation occurs in the testis where sperm is generated. Sperm present in the testis are immature and not ready to fertilize an egg. The second stage is epididymal maturation which occurs in the male tract. After epididymal maturation sperm are not fully mature and are not ready to fertilize an egg. The final stage of maturation is capacitation which occurs in the female tract. After capacitation, sperm are ready to fertilize an egg.

Prior to fertilization after natural mating epididymally mature sperm undergo a final maturation period, capacitation, in the female tract during which they are prepared for interaction with the eggs. Since ejaculated sperm are unable to fertilize eggs immediately on contact with eggs in vitro, capacitation is often considered an essential pre-requisite for the fertilization process. Thus for in vitro fertilization the process is simulated prior to the introduction of the sperm to the egg.

As used herein, the term “capacitation medium” or “capacitating medium” refers to a solution that facilitates capacitation of sperm. A capacitating medium may include a variety of ingredients such as calcium, sodium lactate, sodium pyruvate, HEPES buffer, and sodium bicarbonate and bovine serum albumin among others. An effective capacitation medium for the invention is Human Tubal Fluid (HTS) which is commercially available from sources such as Millipore (EMBRYOMAX® Human Tubal Fluid). In addition the capacitation medium may contain uterine fluid, epididymal fluid, human tubal fluid or synthetic uterine fluid which facilitates capacitation of sperm. Any applicable capacitation medium known to those of skill in the art may be used. As used herein, the term “medium” refers to a solution that facilitates the combining of sperm and either bioactive molecules or lipid carriers. Any applicable medium known to those of skill in the art may be used.

Further, in vitro capacitation is known to occur under certain specified conditions which include a sterile environment, capacitating medium, 37° C., and an atmosphere of reduced O₂. The period of sperm capacitation varies with the species. For example, in the mouse, in vitro capacitation generally takes 45 to 60 minutes in the above conditions.

In example 3, we conveniently combine uptake of epididymal proteins, including SPAM 1, with in vitro capacitation of epididymally mature caudal sperm to enhance the fertilizing capacity of sperm.

Cell-to-cell transfer of glycosyl phosphatidylinositol (GPI)-linked membrane proteins in vivo is known thus far for sperm and erythrocytes, cell types in which biosynthetic ability is absent or limited. This transfer plays a pivotal role in the remodeling of the sperm plasma membrane (PM) during the sperm's maturation in both the male and female genital tracts.

Although sperm leaving the testis are incapable of transcriptional and translational activity, their surface proteins undergo a remarkable degree of modification during epididymal maturation and capacitation in the female tract. During epididymal transit (which may vary from 3-12 days depending on the species) sperm are in an intimate association with the epididymal epithelium and its secretions and thereby exposed to variety of macromolecules that are sequentially added to their PM surface. After epididymal transit, however, sperm are not fully mature and ready to fertilize an egg. In the female, molecules are added to sperm from the secretions of the female tract, where sperm reside for a shorter period. Some of these modifications on the sperm surface result from exchanges between soluble lipid donors or acceptors and the PM, and a variety of the proteins involved are GPI-linked. After capacitation in the female tract, sperm are fully mature and ready to fertilize an egg.

Sperm surface remodeling plays an important role in fertilization. The addition of bioactive molecules on the surface of sperm furthers post-testicular maturation. This remodeling increases the likelihood of successful fertilization with an egg. Deficiencies in sperm surface remodeling, likewise, lead to a reduction in egg fertilization. The inventors have discovered a new composition and method of enhancing fertilization by promoting the remodeling of the sperm surface and thus empowering the sperm to fertilize.

Bioactive molecules of the invention that enhance fertilization by attachment to the sperm surface include, for example, GPI-linked proteins, enzymes, adhesion molecules, immune proteins, antigens and glycoproteins.

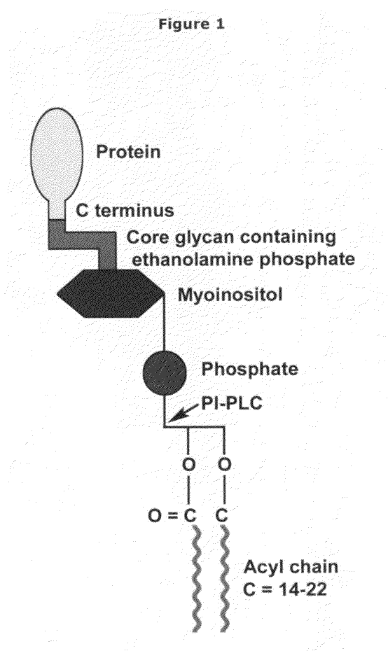

GPI-linked proteins include membrane-associated enzymes and adhesion molecules, among a variety of other glycoproteins. They are anchored to PMs post-translationally via a covalent attachment of glycosylated phosphatidylinositol molecules (FIG. 1) and are confined to the outer leaflet of the lipid bilayer, usually in microdomains which are rich in glycosphingolipids and cholesterol. Some GPI-linked proteins are associated with exosomes or vesicles called epididymosomes which are characterized by a high cholesterol/phospholipid ratio, and many are associated with germ cells. Others are released by apocrine secretion resulting from blebbing of the epithelial lining. Preferred examples of GPI-linked proteins include SPAM1, P34H, CD52, CD55, CD59, and CD73.

As seen in FIG. 1, after triggering the acrosome reaction and the secondary binding of sperm to the zona pellucida, early steps in fertilization, GPI-linked proteins are cleaved in the glycan core by angiotensin-converting enzyme (ACE), an endomannosidase, from the sperm tail's midpiece. This cleavage facilitates further sperm-egg interaction by functional activation of the proteins or removal of the physical barrier they represent to sperm-egg interaction (Kondoh et al., 2005). The addition of surface proteins to the sperm surface by an attachment other than the GPI linkage results in only limited functional activity and maybe counterproductive if it is not able to be cleaved by ACE.

A large number of GPI-linked proteins are involved in reproduction. GPI-linked proteins that were initially shown to be acquired by post-testicular sperm in vivo were ones that were also found on cells in the immune system (e.g. CDS2, CD55, CD59, CD73); thus they were thought to be involved solely in protecting sperm from immune attack in the male and female tract, However, it has now become clear that the distribution/translocation of GPI-linked proteins on the sperm PM during post-testicular maturation underscores the importance of this type of PM attachment directly in the mammalian reproductive process. Facilitated by their unhindered lateral mobility, these proteins are known to participate in epididymal maturation, the signal transduction process in capacitation, acrosomal exocytosis, and sperm-egg interaction. Compared to other types of PM attachments for sperm proteins, the GPI-anchor offers special structural and functional advantages. It facilitates lateral diffusion which not only economizes on the number of required molecules, but improves the dispersion and interaction with other molecules on the sperm PM.

Recently, three fertility centers in Canada showed that a lack of protein P34H, known to be involved in sperm-egg interactions, can be used as a predictor of cases of failed fertilization treatments. (Moskovtsev S. I., et al. Epididymal P34H protein deficiency in men evaluated for infertility. Fertil Steril. 2007 Apr. 13. and Boue F., et al. Cases of human infertility are associated with the absence of P34H an epididymal sperm antigen. Biol Reprod 54:1018-1024, 1996). P34H is another GPI-linked protein that may be used within the present invention to enhance sperm.

During sperm surface remodeling, there is the loss of surface proteins and the selective absorption of epididymal, uterine and oviductal factors on the PM. In the female tract, cholesterol efflux from the sperm PM is known to play an important role in capacitation. Although the mechanism of the efflux is not well understood, there is convincing evidence for the involvement of high density lipoprotein (HDL) and other lipid complexes which serve as acceptors of sperm cholesterol and phospholipids. APOJ/Clusterin and ApoA-1 are implicated in the process of lipid exchange from the sperm PM to epithelial cells of the epididymis and uterus.

Clusterin is a family of multifunctional secretory glycoprotein that is expressed is a variety of body fluids. Some examples of clusterin glycoproteins include APOJ, SGP2, TRPM, gp80 and SP-40. It is known as a chaperone-like protein that can bind lipids and membrane-active proteins and is abundantly expressed in testis (specifically Sertoli cells), epididymis and in the female genital tract, although its specific function has long been the subject of much speculation. Importantly, it is expressed on the surface of sperm and due to its abundance and spatial expression pattern is thought to play an important role in sperm development and maturation. A major fraction of APOJ in the ELF is free or loosely associated with sperm while a smaller fraction is tightly associated with the lipid bilayer. Further, epididymal APOJ forms complexes with other proteins and or/lipids, but not specifically ApoA-1. More recently, it has been shown to be involved in lipid exchange in the male tract where the lipidated protein is endocytosed via a receptor-mediated mechanism at the epithelial cell lining Expression of APOJ and its receptor, Megalin (LRP2), in the male parallels that in the female where the receptor is present in the uterine and oviductal epithelia. It is also maximally expressed during estrous and metestrous.

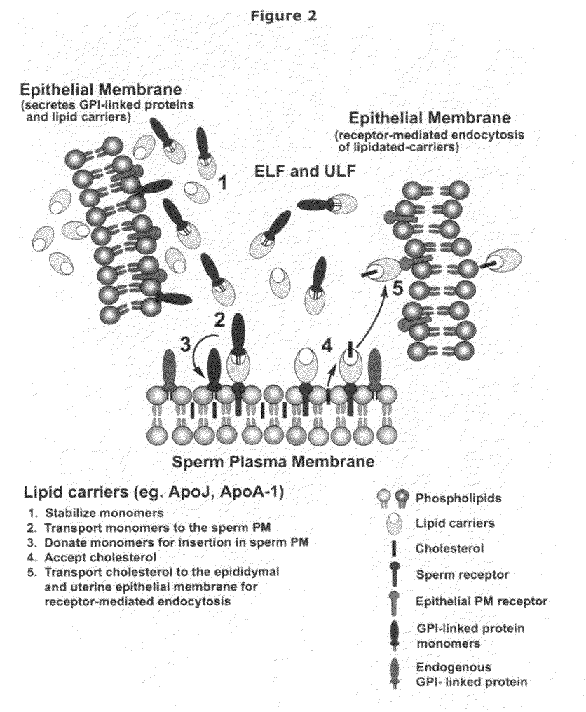

Apolipoprotein A-1 (ApoA-1) is a major protein of plasma HDL and is known to play important roles in lipid transport and metabolism. It has also been shown to bind to a family of bovine seminal plasma proteins. Like APOJ, it is also expressed in the male and female where it is implicated in the process of lipid exchange from the sperm PM to that of the epithelial cells. It shares with APOJ the same receptor (Megalin) and along with a co-receptor, Cubulin, it mediates endocytotic removal of lipidated proteins. While APOJ has been demonstrated to bind to the sperm surface, this has not been clearly shown for ApoA-1. FIG. 2 is a model showing lipid exchange at the surface of sperm within the epididymis and uterus.

Mammalian epididymal luminal fluid (ELF) has been shown to be a complex consisting of particulate membranous vesicles and soluble membrane-free components. This has also been shown to be characteristic of uterine luminal fluid (ULF) However, capacitation takes place in ULF or simulated ULF. Simulated ULF may contain ELF. Importantly, Sperm adhesion molecule 1 (SPAM1), among a number of other GPI-linked proteins present in mouse ELF and ULF, can be acquired on the sperm surface in vitro from both components, with uptake being more efficient from the soluble membrane-free fraction. Sub-fractionation of this soluble component by ultracentrifugation (230,000×g) revealed the presence of oligomeric aggregates in the pellet and predominantly soluble SPAM1 monomers (67 kDa). It has also been shown that SPAM1 uptake from this sub-fraction is modulated by the presence of added exogenous lipoproteins: there was found to be an inverse relationship between the concentration of lipoproteins and SPAM1 transfer to the sperm surface.

SPAM1 is an ideal model for elucidating the mechanisms of sperm uptake and removal of GPI-linked proteins. The specific function(s) of most of the GPI-linked proteins acquired by sperm are unknown. However SPAM1, which is the major mammalian sperm hyaluronidase, plays multifunctional roles in fertilization and is ideal for the studies proposed. Our lab has shown it to be a secretory protein in the epididymides of humans, macaques, rats and mice, and expression appears to be conserved. In mice it has been shown to be expressed in all three regions (the efferent ducts, epididymis, and vas deferens) of the male tract, as well as the accessory organs (prostate, and seminal vesicles). The secretions from all three regions (caput, corpus, cauda) of the mouse epididymis were shown to contain SPAM1 in both a soluble (120S) and vesicular form (120P) (40:60), with the latter having an intact GPI anchor. More recently it has been shown that when Spam1 null sperm are exposed in vitro to unfractionated ELF there was considerable acquisition of SPAM1 and this was accompanied by a significant increase in cumulus penetration. This suggests that epididymal SPAM1 plays a role in sperm PM remodeling and is a marker sperm maturation.

Our lab has shown that SPAM1 is also expressed in all three regions (vagina, uterus, oviduct) of the female genital tract cyclically. It is present predominantly during estrus and is located in both the glandular and the secretory epithelium. More recently, it has been shown that it is secreted in the ULF in both a soluble and a vesicular form, and is also present in the oviductal fluid. Importantly, in vitro SPAM1 uptake by Spam1 null sperm from unfractionated wild type (WT) ULF showed a localization that mimicked that of WT mature sperm, as was the case for uptake from ELF. It is interesting that SPAM1 is associated with lipid rafts which are rich in cholesterol and GPI-linked proteins. It should be noted that lipoproteins such as APOJ could function efficiently in donating their stabilized GPI-linked proteins in the same location that they remove cholesterol.

Soluble lipid carriers, thought to play a role in cholesterol efflux from the sperm plasma membrane, are also responsible for stabilizing soluble GPI-linked monomers and facilitating their insertion via their acyl chains into the outer leaflet of the lipid bilayer. In vitro acquisition of SPAM1 on the surface of caudal mouse sperm from the membrane-free monomeric component of both ELF and ULF is dependent on the presence of Clusterin/APOJ, a lipid carrier abundantly expressed in the genital tracts. When APOJ in ELF and ULF was antibody-inhibited in the soluble monomeric sub-fraction, SPAM1 uptake on mouse sperm was markedly reduced.

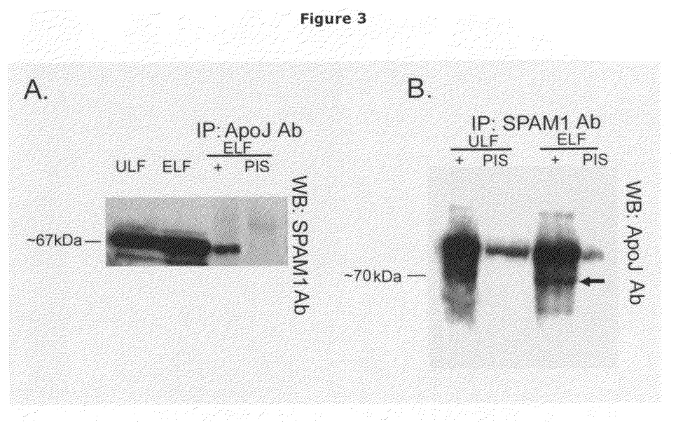

In addition, we have shown an association of SPAM1 and APOJ in immunoprecipitations from the luminal fluids, reflecting the intimate interaction of these proteins. APOJ is known to bind to the sperm surface. In FIG. 3 is shown a Western blot that indicates reciprocal co-immunoprecipitation of SPAM1 and APOJ. This finding reveals that the proteins have an association which is likely mediated by hydrophobic interactions. Such interactions identify a role for APOJ in the transfer of SPAM1 and other GPI-linked proteins from LFs to the sperm plasma membrane. This is the first identified interaction between SPAM1 and APOJ. Interestingly, epididymal soluble prion protein which is GPI-linked was recently shown to form complexes with APOJ. (Ecroyd, H., et al. The epididymal soluble prion protein forms a high-molecular-mass complex in association with hydrophobic proteins. Biochem J 392: 211-219, 2005).

APOJ in ELF and ULF stabilizes monomers of GPI-linked proteins, transports them to the sperm surface where they are inserted into the plasma membrane during epididymal maturation and capacitation. This model extends the currently held view that during cholesterol efflux at the sperm membrane lipid-poor APOJ accepts cholesterol and transports it the epididymal and uterine epithelial membranes for receptor-mediated endocytosis. Our work shows a novel role for APOJ whose exact function has been an enigma for some time. It also has the potential of leading to advances in technology for the delivery of biologically or biomedically relevant membrane-free GPI-linked molecules to the sperm surface before IUI or IVF, to enhance sperm maturation and function. The present invention also extends beyond the reproductive field.

The advantage is that the acquisition of these proteins occurs from membrane-free molecules rather than membranous vesicles. These membrane-free molecules, as well as Clusterin, can be made recombinantly, and used for in vitro interaction with the sperm surface.

This invention deals with an understanding of the physical and chemical interactions that determine the precise delivery of GPI-linked molecules in vitro to the sperm plasma membrane. We have determined that delivery is most efficient from monomers compared to vesicles or oligomeric aggregates, and that delivery of these monomers is enhanced in the presence of at least one lipid carrier, Clusterin or APOJ. SPAM1 is present in low (monomeric) and high (oligomeric) MW complexes. The latter are incapable of transferring SPAM1 and may serve as reservoirs that produce monomers. APOJ has long been known to be present in abundant quantities in the male and female tracts and to be a chaperone molecule. Its precise function has not been clearly delineated, although it is thought to help to bring about the net efflux of cholesterol that occurs at the sperm surface during their maturation in the male and female environments. It is thought to act as an acceptor of cholesterol which is then disposed of at the epithelial membrane lining the epididymal and uterine tract by a process of receptor-mediated exocytosis.

We have found that when Clusterin binds to the sperm membrane it also acts as a donor of lipid molecules to the sperm surface. Using the SPAM1 model, we have shown that antibody blockage of APOJ in the luminal fluid from both the male and female tract considerably inhibits the uptake of this protein. We have also shown that when various amounts of exogenous lipoproteins were added to the soluble fraction of the epididymal luminal fluid there was an inverse relationship between concentration and SPAM1 transfer to the sperm plasma membrane, implicating the involvement of lipoproteins in general in the delivery of GPI-linked proteins.

To confirm the involvement of APOJ in the transfer of GPI-linked proteins to the sperm surface, we used immunoprecipitation to show an intimate association between SPAM1 and APOJ and vice versa (See Example 5). This is the first reported interaction between clusterin/APOJ and SPAM1 in both the epididymal luminal fluid (ELF) and the uterine luminal fluid (ULF), Based on the large number of GPI-linked proteins involved in reproduction, Clusterin is likely to play an important role in the uptake of proteins from the liquid phase of the luminal fluids.

Alternative uses of the invention included, but are not limited to, a method for GPI-transfer technology to express on the cell surface biologically important molecules, might be useful in a variety of ways, e.g. anticancer and antiviral immunotherapy. There are also implications that include disease transmission with respect to prions which have GPI anchors and are known to be added to the sperm surface at ejaculation in rams.

From a theoretical or fundamental point of view, it significantly increases the understanding of the coupled processes of epididymal sperm maturation and capacitation, with respect to the acquisition of GPI-linked proteins in the remodeling of the sperm PM. We have found a novel lipid donor and stabilizer role for the well-known lipid acceptors, APOJ and ApoA-1, known to be involved in sperm maturation, in that they could stabilize monomers of SPAM1 and other GPI-linked proteins in the LFs, and deposit them at the sperm PM for insertion prior to removing cholesterol. Thus, the invention increases the understanding of sperm surface lipid exchange involving the net efflux that occurs during sperm maturation. Specifically, it reveals a more efficient interaction of lipid acceptors and the sperm PM than previously envisaged in cholesterol efflux.

The invention provides a means of adding bioactive molecules, such as SPAM1, P34H or other GPI-linked proteins, to the surface of sperm during the processing that precludes both intrauterine insemination (IUI) and in vitro fertilization (IVF). Prior to the present invention, the only recombinant source of SPAM1 available was a recombinant SPAM1 without the GPI-link or anchor. Human recombinant SPAM1 without the GPI-link/anchor was shown to be 10× more effective than slaughterhouse-derived SPAM1 in the dissolution of the cumulus cells, when mixed with sperm in IVF. (Bookbinder, L. H., et al. A recombinant human enzyme for enhanced interstitial transport of therapeutics. J. Control Release 114: 230-241, 2906 and Kunda, A., et al. Dispersion of cumulus matrix with a highly purified recombinant human hyaluronidase (rHuPH20). Hyaluronan 2003, The Cleveland clinic and Matrix Biology Institute, Poster Session #8, October 11-16, Cleveland, Ohio.). SPAM1 without the GPI-link could be produced recombinantly because it was removable (i.e. solubilized) from the cell by known techniques. Conversely, recombinant synthesis of SPAM1 with the GPI-link intact cannot be removed or solubilized from the cell by known techniques. The GPI-link, however, is necessary for SPAM1 to attach to sperm. The development of a recombinant SPAM1 with anchor intact would be significantly advantageous as sperm acquisition of such a recombinant GPI-linked SPAM1, in addition to increasing cumulus penetration, enhances the signaling involved in acrosomal exocytosis and zona binding, functions unattainable with the current soluble recombinant protein having no lipid anchor.

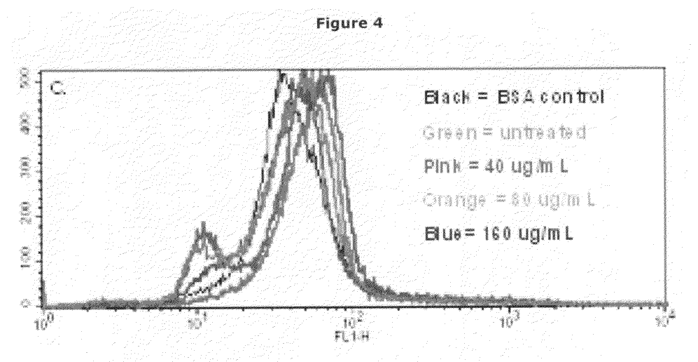

The present invention provides the technology of obtaining such a superior human recombinant SPAM1 with an intact GPI anchor for use in IUI and IVF. We have discovered that clusterin, when added to epididymal proteins at high levels, inhibits the uptake of SPAM1. At high levels, clusterin can remove SPAM1 and other GPI anchored proteins from the cell surface. High levels are considered to be about at least 40 ug/mL of lipoproteins comprising clusterin. Preferred high levels of lipoproteins comprising lipid carrier are about 40 to about 2,000 ug/mL. More preferably, high levels of lipoproteins comprising lipid carrier are about 100 to about 1,000 ug/mL. Effective removal of GPI-linked protein was performed using 800 ug/mL of lipoproteins comprising clusterin. In addition, FIG. 4 shows that SPAM1 uptake is hindered by increasing the lipid carrier concentration.

Similarly, the invention may be used to supply bioactive molecules to patients in whom a lack of bioactive molecule on the sperm surface is detected. Based on the large number of GPI-linked proteins involved in reproduction the present invention is expected to have a far-reaching impact on the reproductive field.

The method of delivery of these bioactive molecules, such as P34H and SPAM1, to the sperm surface is non-invasive. In IUI, recombinant bioactive molecule, such as SPAM1 or P34H, along with recombinant carrier, such as APOJ can be added to the insemination media, such as up to 60 minutes while in the catheter bag, prior to insemination. In the case of IVF fresh or frozen sperm that have undergone purification, such as by Pure Sperm Separation, can be treated with recombinant GPI-linked proteins along with a carrier, such as APOJ, prior to being placed in human tubal fluid and before the final wash after which they are placed in culture medium for inseminating oocytes.

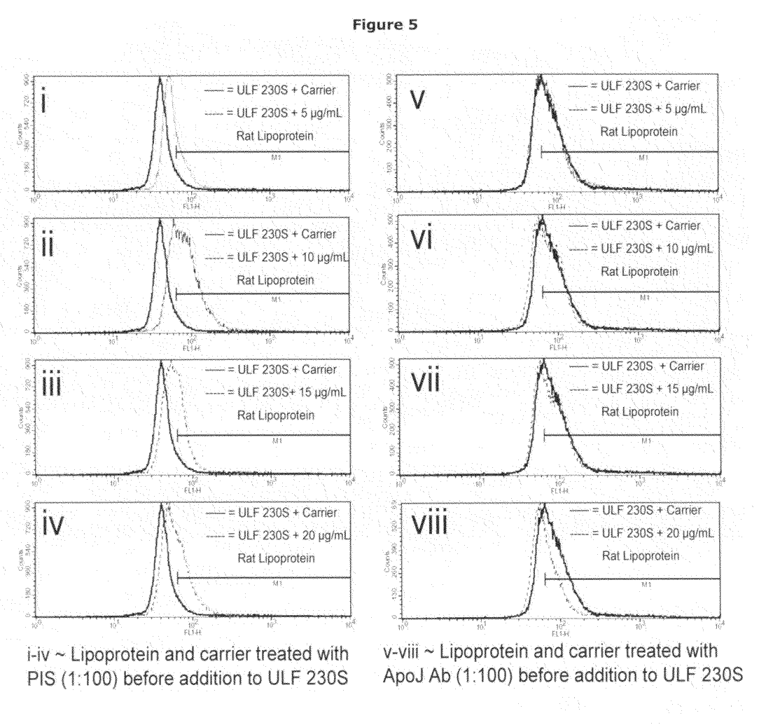

FIG. 5 shows that low concentrations of lipoprotein supplements in uterine luminal fluid (ULF) enhance the uptake of SPAM1 in flow cytometric analysis. Sperm uptake of SPAM1 from the soluble ULF fraction was dramatically enhanced when rat serum lipoproteins (mixed with preimmune serum (PIS) (1:100)) were added at a final concentration of 5-20 pg/mL prior to incubating sperm, as demonstrated by a peak shift to the right (i-iv), when compared to the carrier control. Under identical conditions, this enhancement was negated when APOJ Ab (1:100) was added to the lipoproteins rather than PIS to block APOJ, as demonstrated by the absence of a peak shift to the right (v-viii).

The invention may also be used to remove bioactive molecules from a first amount or aliquot of sperm and to delivery the same bioactive molecules to a second amount or aliquot of sperm. In this embodiment, the bioactive molecules from the first aliquot of sperm are used to enhance sperm maturation and function of the second aliquot of sperm that may, for example, lack a sufficient amount of the bioactive molecule on the sperm surface to effect fertilization of an egg.

In one embodiment, the present invention is directed to a method for enhancing sperm maturation and function comprising, in an in vitro environment: (a) obtaining a first sample of sperm from a first male candidate; (b) treating said first sample of sperm with a solution comprising lipoproteins, wherein the solution comprises an effective amount of lipoproteins to remove at least one bioactive molecule from said first sample of sperm; (c) recovering a supernatant from the treated solution, wherein said supernatant comprises said at least one bioactive molecule and said lipoproteins; and (d) adding said recovered supernatant to a second sample of sperm, whereby said at least one bioactive molecule is transferred to the surface of a sperm.

In one embodiment, the present invention is directed to a method for enhancing sperm maturation and function comprising, in an in vitro environment (a) obtaining a first amount of sperm from a male candidate; (b) adding to said first amount of sperm a solution comprising lipoproteins, wherein the solution comprises at least 40 ug/ml of lipoproteins whereby said at least about 40 ug/ml of lipoproteins removes at least one bioactive molecule (e.g. a GPI-linked protein) from said first amount of sperm; (c) recovering a supernatant from the solution, wherein said supernatant comprises said at least one bioactive molecule and said at least 40 ug/ml of lipoproteins, and (d) adding said supernatant to a medium comprising a second amount of sperm from a second male candidate, wherein said medium comprises less than about 40 ug/ml of lipoproteins and said at least one bioactive molecule is transferred from the medium to the surface of a sperm.

As described in Example 10, the concentrations of purified recombinant APOJ required to effectively remove at least one bioactive molecule from cells as well as transfer at least one bioactive molecule to cells has been determined. Accordingly, in another embodiment, the present invention is directed to a method for enhancing sperm maturation and function comprising, in an in vitro environment (a) obtaining a first amount of sperm from a male candidate; (b) adding to said first amount of sperm a solution comprising APOJ, wherein the solution comprises at least about 150 ng/ml of APOJ whereby said at least about 150 ng/ml of APOJ removes at least one bioactive molecule (e.g. a GPI-linked protein) from said first amount of sperm; (c) recovering a supernatant from the solution, wherein said supernatant comprises said at least one bioactive molecule and said at least about 150 ng/ml of APOJ; and (d) adding said supernatant to a medium comprising a second amount of sperm from a second male candidate, wherein said medium comprises less than about 150 ng/ml of APOJ and said at least one bioactive molecule is transferred from the medium to the surface of a sperm.

The first and second aliquots of sperm may be from the same or from different male candidates. When the first and second aliquots of sperm are from the same male candidate, the resulting enhanced sperm provokes a reduced immunological response upon introduction to the egg and/or uterine tract. Because of the reduced immunological response, sperm enhanced with bioactive molecules from the same male candidate are better able to effect fertilization of an egg.

Multiple sperm samples may be used as the first aliquot of sperm. The multiple sperm samples may have been produced in the same ejaculate, or from different ejaculates of the same or of different male candidates. The multiple sperm samples may be combined into a single first aliquot of sperm. Alternatively, each individual sperm sample may be individually processed to removed their respective bioactive molecules. Thereafter, the removed bioactive molecule samples may then be combined for transfer to a second aliquot of sperm or used individually for transfer to a second aliquot of sperm. The second aliquot of sperm may be a sub-set of the first aliquot(s) of sperm.

The present invention may also include isolation/purification and/or dilution steps. For example, the bioactive molecules removed from a first amount of sperm may be separated from the high lipoproteins or APOJ concentration environment. Any isolation, purification or separation method known in the art may be used to remove the bioactive molecules from the high concentration solution. Thereafter, the separated bioactive molecules may be re-solubilized or diluted in a solution or medium comprising a lower concentration of lipoproteins or APOJ for transfer to a second aliquot of sperm. Alternatively, the bioactive molecules removed from a first amount of sperm and contained in a high lipoproteins or APOJ concentration environment may be diluted to a lower concentration of lipoproteins or APOJ concentration. The lower concentration may be a concentration when added to a medium comprising a second amount of sperm from a second male candidate, the medium comprises less than about 40 ug/ml of lipoproteins or of less than about 150 ng/ml of APOJ.

As described herein, the methods of enhancing the maturation and function of sperm by removal and addition of bioactive molecules from different aliquots of sperm may further comprise delivering said second amount of sperm (i.e. enhanced sperm) to a uterine tract. Alternatively, the methods may further comprise adding at least one egg to the medium comprising said second amount of sperm (i.e. enhanced sperm) and incubating until said egg is fertilized. The methods may further comprise cultivating said fertilized egg into an embryo. Finally, the methods may further comprise delivering said embryo to a uterine tract.

It is important to understand that lipid carriers, such as APOJ/clusterin, that are effective in the invention, are preferably membrane free, and, as such it is certainly anticipated, and well within the means of those having skill in the art, that recombinant means can be used to promote the process of the invention.

Compositions comprising a substantially purified bioactive molecule and a lipid carrier for administration to animals, can be prepared by techniques known to those skilled in the art. For example, a purified preparation can be obtained following an individual technique or a series of preparative or biochemical techniques. The procedures can include, for example, but are not limited to, ammonium sulfate fractionation, gel filtration, ion exchange chromatography, affinity chromatography, density gradient centrifugation and electrophoresis. Recombinant proteins can be made by a variety of methods including but not limited to transformation, phage introduction, and non-bacterial transformation.

One method of preparation of a substantially purified bioactive molecule or lipid carrier of the invention is using recombinant means. Recombinant bloactive molecules, including GPI-linked proteins, and lipid carriers may be produced and purified by known techniques, such as those described in US Publication Nos. 2004/0268425 and 2007/0197466. The entirety of both references are herein incorporated by reference.

For example, one aspect of the invention pertains to vectors, containing the sequence encoding the desired protein of the invention, for example, a nucleic acid encoding a bioactive molecule, such as GPI-linked protein or a lipid carrier such as clusterin or derivatives thereof for its convenient cloning, amplification, and/or transcription. As used herein, the term “vector” refers to a nucleic acid molecule capable of transporting another nucleic acid to which it has been “operably linked.” One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the transcription of sequences to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), and artificial chromosomes, which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be transcribed. Within a recombinant expression vector, operably-linked is intended to mean that the nucleotide sequence of interest is linked to the regulatory sequence(s) in a manner that allows for transcription and/or expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of transcription, and/or expression of protein desired, etc. The expression vectors of the invention can be introduced into host cells to thereby produce proteins or peptides, including fusion proteins or peptides, encoded by nucleic acids as described herein. The recombinant expression vectors of the invention can be designed for transcription and/or expression in prokaryotic or eukaryotic cells. For example, transcription and/or expression in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and/or translated in vitro, for example using T7 promoter regulatory sequences and T7 polymerase.

In another embodiment, the recombinant vector is capable of directing transcription of the sequence encoding the desired protein preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al, 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the alpha-fetoprotein promoter (Campes and Tilghman, 1989. Genes Dev. 3: 537-546).

In other aspects, the invention relates to a host cell comprising the sequence encoding the desired protein of the invention. In certain embodiments, the host cell comprises a vector, plasmid or artificial chromosome nucleic acid containing one or more transcription regulatory nucleic acid sequences operably linked with the sequence encoding the desired protein of the invention. The vector or plasmid nucleic acids can be, for example, suitable for eukaryotic or prokaryotic cloning, amplification, or transcription. In other embodiments, the invention comprises a plurality of aptameric GRO sequences linked contiguously as a single polynucleotide chain. In still other embodiments, the invention comprises a nucleic acid vector containing a plurality the sequences encoding the desired protein linked contiguously and operably linked with the nucleic acid sequence of the vector.

The term “host cell” includes a cell that might be used to carry a heterologous nucleic acid, or expresses a peptide or protein encoded by a heterologous nucleic acid. A host cell can contain genes that are not found within the native (non-recombinant) form of the cell, genes found in the native form of the cell where the genes are modified and re-introduced into the cell by artificial means, or a nucleic acid endogenous to the cell that has been artificially modified without removing the nucleic acid from the cell. A host cell may be eukaryotic or prokaryotic. For example, bacteria cells may be used to carry or clone nucleic acid sequences or express polypeptides. General growth conditions necessary for the culture of bacteria can be found in texts such as BERGEY'S MANUAL OF SYSTEMATIC BACTERIOLOGY, Vol. 1, N. R. Krieg, ed., Williams and Wilkins, Baltimore/London (1984). A “host cell” can also be one in which the endogenous genes or promoters or both have been modified to produce the sequence encoding the desired protein of the invention.

Protein purification can be preformed by any method known to one of skill in the art. These methods include extraction, precipitation and differential solubilization, ultracentrifugation and chromatographic methods such as size exclusion chromatography, separation based on charge or hydrophobicity, ion exchange chromatography, affinity chromatography, metal binding, and immunoaffinity chromatography. Purification may be preparative or analytical.

Extraction: Depending on the source, the protein is brought into solution by breaking the tissue or cells containing it by several known methods, such as repeated freezing and thawing, sonication, homogenization by high pressure or permeabilization by organic solvents. After this extraction process soluble proteins may be in the solvent, and can be separated from cell membranes, DNA etc. by centrifugation.

Precipitation and differential solubilization: In bulk protein purification, protein are isolated by precipitation with ammonium sulfate. This is performed by adding increasing amounts of ammonium sulfate and collecting the different fractions of precipitate protein.

Ultracentrifugation: Centrifugation is a process that uses centrifugal force to separate mixtures of particles of varying masses or densities suspended in a liquid. When a vessel (typically a tube or bottle) containing a mixture of proteins or other particulate matter, such as bacterial cells, is rotated at high speeds, the angular momentum yields an outward force to each particle that is proportional to its mass. The tendency of a given particle to move through the liquid because of this force is offset by the resistance the liquid exerts on the particle. The net effect of spinning the sample in a centrifuge is that massive, small, and dense particles move outward faster than less massive particles or particles with more drag in the liquid. When suspensions of particles are spun in a centrifuge, a pellet may form at the bottom of the vessel that is enriched for the most massive particles with low drag in the liquid. The remaining, non-compacted particles still remaining mostly in the liquid are called the supernatant and can be removed from the vessel to separate the supernatant from the pellet. The rate of centrifugation is specified by the angular acceleration applied to the sample, typically measured in comparison to the g. If samples are centrifuged long enough, the particles in the vessel will reach equilibrium wherein the particles accumulate specifically at a point in the vessel where their buoyant density is balanced with centrifugal force. Such an “equilibrium” centrifugation can allow extensive purification of a given particle.

Chromatographic methods: A protein purification protocol may contain one or more chromatographic steps. The basic procedure in chromatography is to flow the solution containing the protein through a column packed with various materials. Different proteins interact differently with the column material, and can thus be separated by the time required to pass the column, or the conditions required to elute the protein from the column. Usually proteins are detected as they are coming off the column by their absorbance at 280 nm. Many different chromatographic methods exist, including size exclusion chromatography, separation based on charge or hydrophobicity, ion exchange chromatography, affinity chromatography, metal binding, and immunoaffinity chromatography.

These compositions can be prepared to deliver an effective amount or dose of bioactive molecule and/or lipid carrier. An effective dose is an amount that is effective in the remodeling of sperm cells. An effective dose is also an amount that is effective in increasing the likelihood of fertilization.

In determining an effective amount or dose of bioactive molecule and/or lipid carrier, a number of factors are considered by the attending diagnostician, including, but not limited to: the species of the mammal; its size, age, and general health; the response of the individual patient or sperm; the particular bioactive molecule administered; the particular carrier administered, the mode of administration; the characteristics of the preparation administered; the dose regimen selected; the use of concomitant medication; and other relevant circumstances.

The composition of the invention can be administered in any form or mode which makes the bioactive molecule and carrier effective. Suitable modes of administration include oral, inhalation, nasal, buccal, topical, rectal, sublingual, transdermal, vaginal, otic, ophthalmic or parenteral administration. Parenteral administration may include intratracheal or inhalant aerosol administration, subcutaneous injection, intravenous injection, intraperitoneal injection, intramuscular injection, intrasternal injection, intrathecal injection, intraventricular and intracerebroventricular injection and infusion techniques. Transdermal and vaginal compositions are generally preferred. One skilled in the art of preparing formulations can readily select the proper form and mode of administration depending upon the relevant circumstances.

A bioactive molecule and carrier of the invention can be administered in the form of pharmaceutical compositions or medicaments which are made by combining a bioactive molecule and a carrier, with pharmaceutically acceptable carriers or excipients, the proportion and nature of which are determined by the chosen route of administration, and standard pharmaceutical practice. The term “pharmaceutically acceptable” refers to a molecular entity or composition that does not produce an allergic or similar unwanted reaction when administered to animals or humans.

The pharmaceutically acceptable carriers used in conjunction with the bioactive molecules and lipid carriers of the present invention vary according to the mode of administration. Solid carriers suitable for use in the composition of the invention include one or more substances which may also act as flavoring agents, lubricants, solubilizers, suspending agents, fillers, glidants, compression aides, binders, tablet-disintegrating agents or encapsulating materials. In powders, the carrier may be a finely divided solid forming an admixture. In tablets, the carrier may be mixed to provide the necessary compression properties in suitable proportions and compacted in the shape and size desired. Solid carriers suitable for use in the composition of the invention include calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers suitable for preparing solutions, suspensions, and emulsions may be employed in the composition of the invention. The actives may be dissolved or suspended in a pharmaceutically acceptable liquid carrier such as water, an organic solvent, or a pharmaceutically acceptable oil or fat, or a mixture thereof. Said liquid composition may contain other suitable pharmaceutical additives such as solubilizers, emulsifiers, buffers, preservatives, sweeteners, flavoring agents, suspending agents, thickening agents, coloring agents, viscosity regulators, stabilizers, osmo-regulators, or the like.

The compositions or medicaments are prepared in a manner well known in the pharmaceutical art. The carrier or excipient may be a solid, semi-solid, or liquid material that can serve as a vehicle or medium for the active ingredient. Suitable carriers or excipients are well known in the art.

The concentration of bioactive molecule and carrier can vary widely as a function of the age, weight and state of health of the patient, the nature and level of need for sperm enhancement, as well as of the administration route. These concentration ranges can naturally be adjusted for each patient according to the results observed. The percentage of bioactive molecule in the composition or present in the medium or capacitation mediums may range from about 0.01% to about 99.9%. The percentage of lipid carrier in the composition or present in the medium or capacitation mediums may range from about 0.01% to about 99.9%.

### EXAMPLES

### Example 1

For ELF and ULF in vitro transfer of SPAM1 (and a related hyaluronidase, HYAL3) occurs efficiently from both the 120,000×g pellet (120P) vesicles and the soluble 120,000×g supernatant (120S) fractions, with the latter being greater.

### Procedure

ELF was collected from the epididymides of sexually mature males as described, and centrifuged at 16,100×g to pellet cellular fragments and sperm. The supernatant was confirmed to be sperm-free after microscopic examination. ULF collected by flushing uteri from superovulated females, was also clarified via centrifugation. Ultracentrifugation (120,000×g for 2 hr at 4° C.) of the LFs was performed to separate the vesicular (120P) from the soluble (120S) fraction. Caudal sperm were collected as described. They were exposed to unfractionated ULF and ELF and their fractions at a concentration of ˜1 mg/ml protein for 2 hr at 37° C. and 32° C., respectively. After incubation, immunocytochemistry was performed and flow cytometric analysis used to quantify SPAM1 acquisition, as compared to sperm incubated in Bovine Serum Albumin (BSA) or Phosphate Buffered Saline (PBS) (which were negative controls). Similar experiments were performed to test for HYAL3, a related hyaluronidase, in ELF and all experiments were repeated twice.

### Results and Interpretation

Sperm exposed to unfractionated LF acquired considerable amounts of SPAM1 compared to the control. Unexpectedly, sperm exposed to the 120S acquired more SPAM1 than those exposed to the 120P. This was also the case for HYAL3. The findings are similar to those for the intake of GPI-linked proteins by Chinese Hampster Ovary (CHO) cells and RBC incubated in seminal plasma and show that transfer from the soluble phase is more efficient than that from vesicles.

### Example 2

SPAM1 acquisition is targeted to the PM of sperm and not to that of RBC and the localization may pattern may depend on the fraction.

### Introduction and Rationale

Since RBC share with sperm the ability for uptake of GPI-linked proteins, it was important to determine if SPAM1 could also be transferred to their PM. Thus experiments were performed using mouse RBC for uptake from unfractionated ULF under the identical conditions used for sperm binding. Additionally Spam1 null mice were utilized to investigate if localization of uptake on the PM was influenced by the specific fraction of ULF, 120S versus 120P.

### Results and Interpretation

While sperm acquired SPAM1 as demonstrated by an increase in fluorescence intensity, there was no transfer for RBC. Because RBC are known to carry GPI-linked proteins, acquired from the plasma the lack of SPAM1 acquisition in RBC suggests that sperm may have specific lipid raft associated microdomains within the PM for SPAM1 binding or adsorption, or alternatively, that there may be specific sperm receptor(s) that mediate the binding. Immunocytochemical images localize SPAM1, acquired from ULF unfractionated and fractionated, to regions of the sperm PM directly overlying the acrosomal cap, and to the midpiece of the tail. However in a large number of cells, the distribution of SPAM1 was distributed throughout the midpiece of the tail. This distribution pattern suggests that initially there may be random insertion in the PM followed by migration of the protein to the localized areas over the acrosome.

### Example 3

Repeat ultracentrifugation of LFs with increasing force enrich for SPAM1 monomers which are the primary vehicles of transfer in the liquid phase.

### Introduction and Rationale

Although for ELF the proportion of SPAM1 in the 120S and 120P fractions is 40:60, the 120S fraction appears to be more efficient in transferring SPAM1 and HYAL3 to the PM, as is the case for ULF. Membrane-free transfer of GPI-linked proteins has been documented from seminal plasma and filtered blood plasma, but to date this is the first demonstration of transfer for membrane-free GPI-linked proteins from the ELF and ULF. Since GPI anchors are highly hydrophobic, multiple GPI-linked molecules are expected to aggregate due to a more favorable level of entropy. Thus, monomers are likely to be in equilibrium with oligomers, with the concentration of the protein determining the proportion of each of these fractions. The equilibrium would shift towards aggregates when the critical micellar concentration is present, and towards monomers when the amount of protein falls below this level. Thus it is important to investigate the physical nature of the soluble SPAM1 in the liquid phase.

### Procedure

ELF was subjected to ultracentrifugation at 120,000×g for 2 hr. The resulting supernatant was centrifuged at 150,000×g for 4 hr. This process was repeated at speeds of 190,000 (8 hr) and 230,000×g (16-24 hr). All pellets were resuspended in the initial volume of 5 ml to determine the relative concentration and form of SPAM1 in each fraction. Equal volumes of each sample were subjected to native PAGE and Western blot analysis.

### Results and Interpretation

Both monomeric and high MW forms of SPAM1 were detected for each fraction, however the proportion of each varied among fractions. Monomeric SPAM1 (67 kDa) was relatively enhanced with sequential repeat utracentrifugation and was most abundant in the 230S fraction. This indicates that either removal of SPAM1 drops its concentration below that of the critical micellar concentration necessary for aggregation of monomers (high MW smears), or that oligomeric SPAM1 can be pelleted via ultracentrifugation. These results also demonstrate that LF 230S is monomer-rich.

### Example 4

Characterization of the Monomer-rich fraction (230S) and its ability to Transfer SPAM1 in LFs Rationale.

Since GPI anchors are highly hydrophobic, the transport of membrane-free GPI-linked molecules within an aqueous solution is highly unlikely without an amphipathic carrier. It is proposed, as depicted in FIG. 2, that lipoproteins which are abundant in the LFs could function as carriers of these proteins since they are well-known acceptors for cholesterol. Thus, the affinity of the monomer-rich 230S fraction will be determined, among the others, for lipoproteins. Then it will be investigated if it can transfer SPAM1, and its efficiency in doing so relative to the 230P fraction. Finally, it will be determined how exogenous lipoproteins might affect the ability of the 230S fraction to transfer SPAM1.

### Procedure

Fractions separated in Example 3 were subjected to native gel electrophoresis to detect their association with lipoproteins, using a rat anti-HDL antibody (prepared by Prof. David Usher in our Department), with a broad specificity for HDL, ApoA-1, and ApoE for Western analysis.

ELF 120S from mature males was subjected to ultracentrifugation at 230,000×g for 2 hr to pellet all membranous vesicles. Caudal sperm were incubated in ELF 230S, ELF 230P or BSA under aforementioned conditions. After incubation, sperm were immunostained for SPAM1 and analyzed for SPAM1 uptake via flow cytometry.

Lipoproteins were isolated by density ultra-centrifugation from rat serum. ELF 230S samples were treated with increasing concentrations of rat lipoproteins before incubation with caudal sperm; sperm incubated in NaCl carrier and BSA were used as a control. Sperm were analyzed for SPAM1 acquisition via flow cytometry.

### Results and Interpretation

Native PAGE gel electrophoresis showed ELF supernatants to be more highly associated with lipoproteins than were the pellets. Western analysis of the various fractions of ELF showed no and low association with the pellets at 120P (vesicles) and 230P (aggregates), respectively; but high association with both supernatants, with the 230S being greater than the 120S. Thus there is a direct relationship between the proportion of monomers and the level of associated lipoproteins.

Caudal sperm incubated in ELF 230S acquired demonstrable levels of SPAM1 when compared to those incubated in BSA as determined by an increase in fluorescence intensity, yet those incubated in 230P demonstrated comparatively negligible SPAM1 uptake. This indicates that the primary form of SPAM1 that is transferred to the sperm surface from LF 120S resides in 230S.

Finally, when various amounts of exogenous rat lipoproteins were added to the 230S ELF supernatant SPAM1 transfer to the sperm PM was inhibited in a concentration-dependent manner, implicating the involvement of lipoproteins in transfer. Alternatively, lipoproteins could sequester SPAM1 making it inaccessible for transfer, or saturate monomeric SPAM1 uptake sites on sperm.

### Example 5

APOJ Antibodies inhibit SPAM1 transfer from the 230S fraction in ELF/ULF and co-immunoprecipitation reveals an association of APOJ and SPAM1.

### Introduction

With the results of the previous experiment implicating the involvement of lipoproteins in SPAM1 uptake from the 230S, it was important to block one of our candidates to determine its impact on transfer. Rat APOJ antibody was provided to us from the laboratory of Dr. Michael Griswold, Washington State University for this purpose.

### Procedure

The APOJ antibody (Ab) is polyclonal and was generated in rabbit. Since SPAM1 antibody is also a rabbit polyclonal antibody, it was important to remove the AopJ antibody (Ab) from the sperm after incubation in the 230S before immunodetection of SPAM1. Several dissociating agents at extremes of salinity and pH were tested for their ability to remove APOJ, with 1 M KCl (pH 7.2) giving the best results. With this reagent, virtually all of the APOJ antibody could be stripped from the sperm prior to immunodetection of SPAM1. Thus 1 M KCl was used for all the experiments prior to quantitation of SPAM1 uptake in the presence of APOJ.

WT ELF or ULF was subjected to centrifugation for 3 hr at 230,000×g. Caudal sperm (from the ELF donors) were washed, and incubated in PBS, or LF 230S+preimmune serum (PIS) or 230S+APOJ Ab (both 1:1000) for 2 hr. After uptake, sperm were washed twice in PBS, and subjected to 1M KCl (pH 7.2) for 15 min at RT to remove APOJ Ab bound to the sperm surface. Sperm were then washed 3 times in PBS, and processed for SPAM1 detection with our primary SPAM1 Ab (1:320) and FITC-conjugated secondary Ab (1:400) followed by flow cytometric analysis.

To determine an association of SPAM1 and APOJ, immunoprecipitation was performed on the 230S fraction. ELF/ULF 230S was treated with PIS or SPAM1 Ab (1:1000), overnight, (4° C.). Samples (1 ml) were incubated with 125 μl Seize X Protein A beads (Pierce) overnight at 370C. Beads were washed 3× in 1×PBS and treated with 100 mM DTT in sample loading dye and heated to 60° C. for 5 min. to extract immunoprecipitated proteins. Samples were probed for the presence of SPAM1 and APOJ via Western analysis.

### Results and Interpretation

Data show that there was a remarkable degree of inhibition of SPAM1 uptake from both ELF and ULF. This strongly implicates APOJ in the transfer of SPAM1 from both the epididymal and uterine secretions. Importantly, it also demonstrates an association between SPAM1 and APOJ in the LFs. Western blot analysis indicates that these two proteins can be co-immunoprecipitated. This finding reveals an association of the proteins and suggests that they might be interacting. Such an interaction could identify a role for APOJ in the transfer of SPAM1 and other GPI-linked proteins from ELF and ULF to the sperm PM.

Although the invention is illustrated and described herein with reference to specific embodiments, the invention is not intended to be limited to the details shown. Rather, various modifications may be made in the details within the scope and range of equivalents of the claims and without departing from the invention.

### Example 6

### Lipoprotein Isolation

Rat serum lipoprotein was isolated by density ultracentrifugation. Rat blood was subjected to centrifugation at 2,000×g for 20 min to pellet red blood cells (RBCs). The density of the resulting rat serum was increased to 1.21 g/mL by adding 1.41 g. sodium bromide (NaBr) to a final volume of 5 mL (adjusted with water to a final weight of 6.05 g) and ultracentrifuged at 230,000×g for 48 hr. The protein concentration of the resulting supernatant was determined by a biocinchoninic acid assay (BCA kit, Pierce). It was shown to contain lipoproteins via dot blot analysis for high density lipoprotein (HDL) and APOJ. For clinical trials, purified human APOJ that is commercially available (Millipore) alleviates the need to use the crude lipoprotein extract from rats.

### Example 7

### In Vitro Fertilization

In vitro fertilization will be performed by retrieving egg(s) from the ovary of a female. The eggs will be retrieved using (from) known techniques, such as a transvaginal technique involving an ultrasound-guided needle piercing the vaginal wall to reach the ovaries. The needle follicles will be aspirated, and the follicular fluid handed to the IVF laboratory to identify ova. The retrieval procedure will take about 20 minutes and will usually be done under conscious sedation or general anesthesia. Sperm will be collected from the male using known techniques. An aliquot of about 10⁶ sperm will be combined with about 100 ng/mL to about 100 ug/mL of human APOJ solubilized recombinant SPAM1 protein extract from the surface of CHO cells and about 0.1 to about 100 ug/mL, preferably about 1.0 to about 40 ug/mL and most preferably about 5 to about 20 ug/mL of lipoproteins comprising lipid carrier in conventional in vitro fertilization apparatus. This combination will be performed both in the presence of the egg and outside the presence of the egg. The concentration, time and other conditions used will be optimized to achieve maximum transfer of SPAM1 to the sperm prior to interaction with the egg. When performed outside the presence of the egg, the egg will be introduced to the combined mixture. The combination of egg, sperm, APOJ and SPAM1 will be maintained under normal in vitro fertilization conditions until fertilization of the egg is achieved. After fertilization, the fertilized egg will be cultured under normal culturing conditions until the fertilized egg produces an embryo. Thereafter, the embryo will be transferred using known transfer techniques into the uterus of a female.

### Example 8

### IUI

IUI will be performed by collecting sperm from a male using known techniques. An aliquot of about 10⁶ sperm will be combined with about 100 ng/mL to about 100 ug/mL of human APOJ solubilized recombinant SPAM1 protein extract from the surface of CHO cells and about 0.1 to about 100 ug/mL, preferably about 1.0 to about 40 ug/mL and most preferably about 5 to about 20 ug/mL of lipoproteins comprising lipid carrier in conventional IUI apparatus. The combination of sperm, APOJ and SPAM1 will be maintained under normal IUI conditions. The concentration, time and other conditions used will be optimized to achieve maximum transfer of SPAM1 to the sperm prior to interaction with the egg. Thereafter, the SPAM1 enhanced sperm will be transferred using known transfer techniques into the uterus of a female for fertilization of an egg.

### Example 9

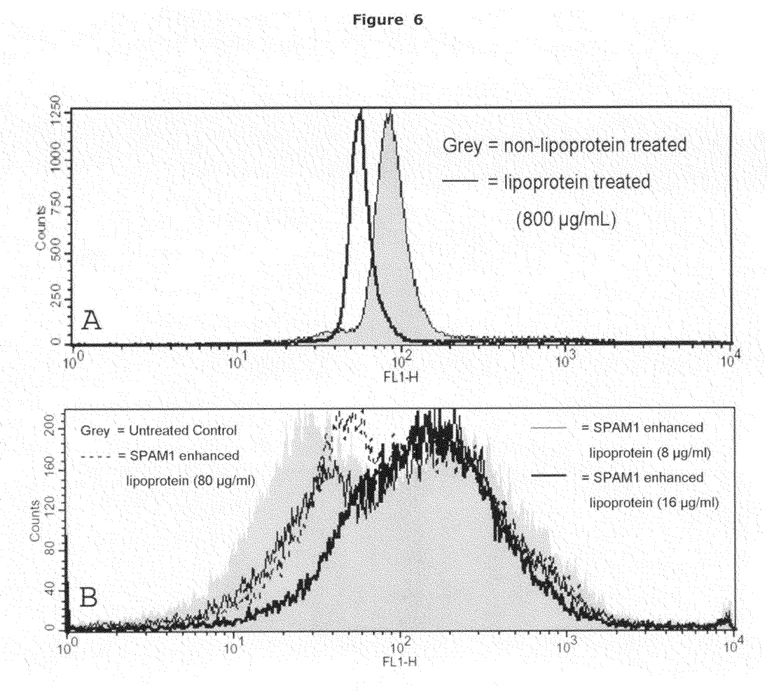

Introduction: Fertilization is dependent on a series of required steps that begin with the penetration of the cumulus matrix by sperm, via their neutral hyaulronidase activity. Since SPAM1 plays a role in several of these steps, it is important to determine if its transfer from the soluble fraction of LFs to the sperm PM increases sperm maturation and fertilizing ability. (See FIG. 6) Functional studies to determine the impact of SPAM1 transfer from the more efficient membrane-free fraction is needed. As a preliminary test, the ability of SPAM1 null sperm to penetrate the cumulus after SPAM1 transfer from unfractionated ELF was assessed. Also assessed was whether murine SPAM1 is involved in HA-enhanced progesterone-induced acrosome reaction, a known functional test for human sperm, in order to determine if the test could be used in AIM III.

Procedure: Working with Dr. Ron Feinberg of the Reproductive Associates of Delaware (Newark, Del.) semen samples were obtained from men undergoing IVF or ISCI, for this purpose. Liquefied semen samples were obtained from the clinic. Sperm were then washed in PBS and proteins extracted in solubilization buffer to determine the level of hyaluronidase activity, using hyaluronic acid (HA) substrate gel electrophoresis. HA Substrate Gel Electrophoresis (HASGE) SPAM1 hyaluronidase activity in sperm protein extracts was measured. Briefly, HA from bovine vitreous humor was added to a 10% SDS-polyacrylamide gel (final concentration 0.3 μg/mL). Gels were loaded with 20 μg of non-reduced proteins and run at 15 mA. After completion, they were incubated in 3% Triton X-100 in PBS for 2 hr at RT, then at 37C for 36 hr in 100 mM sodium acetate (pH 7.0). To visualize digestion of HA, gels were stained with 0.5% alcian blue in 3% acetic acid for 2 hr, and destained in 7% acetic acid until digestion was visible. Gels were counterstained with Coomassie Brilliant Blue G-250 and destained with methanol-acetic acid.

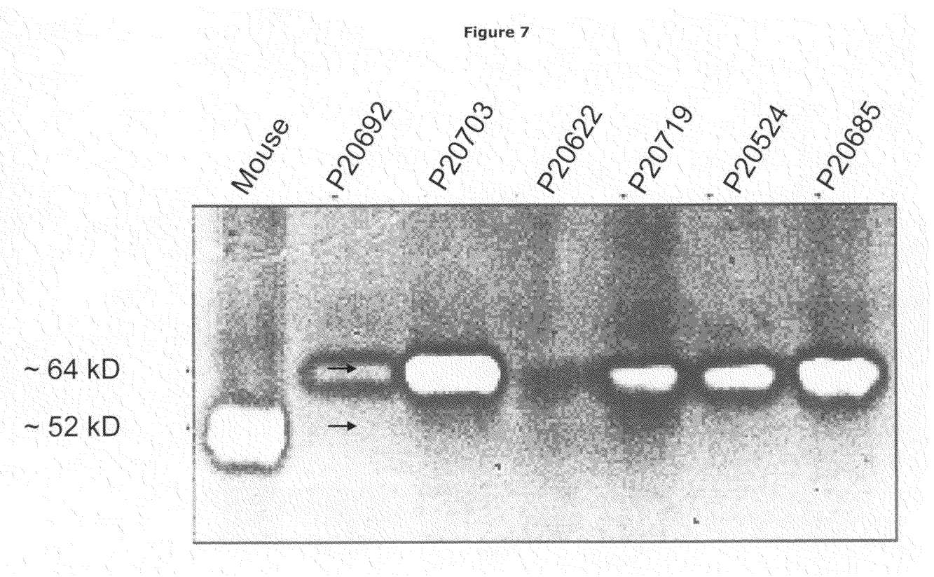

FIG. 7 shows the results from 6 men studied consecutively between June and August in 2006. It is unknown which males are from couples with male- or female-factor infertility. It is evident that there is a large variation in the level of hyaluronidase activity seen in this small sample: ⅙ or 16.6% has no activity and 3/6 had drastically reduced activity. Whether or not these men are representative of the population is also unknown, but the data clearly shows substantial variation. This work suggests that there will be a proportion of males who might benefit from delivery of SPAM1 in vitro during capacitation for IVF or prior to intrauterine insemination, to improve sperm fertilizing ability. Although low hyaluronidase activity might not be equivalent to low SPAM1 protein level, it is likely that there will be some individuals who will have sperm with the capacity to acquire exogenous SPAM1.

### Example 10

### Introduction

During post-testicular maturation of sperm, a variety of GPI-linked proteins are added to the surface of the sperm as they traverse the male and female tract. These proteins include receptors and immunoprotection molecules, and enzymes that participate in both sperm maturation and the fertilization processes. Importantly, the proteins play a role in the remarkable degree of sperm surface modification and remodeling that occur during epididymal maturation and capacitation. One GPI-linked protein that is present in both the epididymal and the uterine tract and has been shown to be added to sperm in vitro is SPAM1. Widely conserved and the major sperm surface hyaluronidase, SPAM1 is a multifunctional protein with essential roles in primate fertilization. The impact of the in vitro uptake of SPAM1 by caudal mouse sperm from ELF and ULF indicates that it is a marker of sperm maturation. Epididymal and uterine SPAM1, which is present during proestrus and estrus, are therefore ideal candidates for investigating the mechanisms of GPI-linked protein transfer to the sperm surface.

GPI-linked proteins are known to exist in LFs in both a vesicular and a soluble membrane-free fraction. Recently we investigated the mechanism of SPAM1 uptake by caudal mouse sperm from the vesicles of ELF (epididymosomes) and ULF (uterosomes), and showed that while uptake occurs from both fractions, it was more efficient from the soluble fraction. Similar to ELF and ULF, the fluid phase of the seminal plasma is known to carry GPI-linked proteins such as CD59, CD55 and CDw52 which are transferred to the human sperm surface. Also, soluble GPI-linked prion protein has been reported to be added to the sperm surface from the seminal plasma at ejaculation. While it has been shown that vesicle docking mediates transfer from the vesicular fraction of LFs, the mechanism involved in the uptake of GPI-linked proteins from the liquid phase of any reproductive fluid has not been elucidated. More importantly, it is unknown how GPI-linked proteins with their lipid anchors are stabilized and solubilized in the aqueous LFs. We thus considered the existence of ideal lipid carriers in the LFs and the seminal plasma.

Several multifunctional secretory lipoproteins, including APOJ, have been detected in the uterus and epididymis. Although its specific function in the epididy mis is still unclear, APOJ, which is a widely expressed and highly conserved chaperone-like protein, is known to bind both lipids and membrane-active proteins in a variety of body fluids. It is therefore an attractive candidate for solubilizing and stabilizing GPI-linked proteins. It also has been detected on the sperm surface and epididymal APOJ forms complexes with other proteins and/or lipids. Further in conjunction with its receptor, Megalin, it is believed to play a role in the lipid exchange accompanying the remodeling of the sperm plasma membrane during sperm maturation in both the male and the female tract. In the latter, Megalin was detected in elevated levels during estrous and proestrous. This parallels the secretion of SPAM1 whose co-expression with APOJ would facilitate their association via hydrophobic interactions. Using SPAMX as a model, the objective of the present investigation was to determine the mechanism involved in the transfer of membrane-free GPI-linked proteins from the fluid phase of LFs to the sperm surface and the role that APOJ may play in mediating the process.

### Materials and Methods

Animals and Reagents: The studies conform to the guide for the Care and Use of Laboratory animals published by the National Institutes of Health (publication 85-23, revised 1985) and were approved by the Animal Care Committee at the University of Delaware. The sexually mature 3-6 month-old male and 4-6 week-old female ICR (Institute of Cancer Research) mice used throughout these studies were obtained from Harlan Sprague-Dawley (Indianapolis, Ind.). The use of human sperm was approved by the Human Subject Review Board of the University of Delaware and informed consent was obtained from the subject studied.

All reagents were purchased from Sigma Chemical Co. (St. Louis, Mo.) unless otherwise specified. The rabbit anti-mouse SPAM1 antiserum used throughout these studies is a polyclonal anti-peptide ((C)NEKGMASRRKESSD in the C-terminus (#381-395)) custom made by Zymed (South San Francisco, Calif.), previously used and shown to be specific for SPAM1 via peptide inhibition. Pre-immune serum (PIS) from the antibody donor was also obtained from Zymed and used as controls to validate the experiments. The rabbit anti-rat APOJ antibody was generously provided by the laboratory of Dr. Michael Griswold (Washington State University). The rabbit polyclonal anti-macaque antibody used in these studies to analyze human sperm SPAM1 was generated against the recombinant protein and was generously provided by the laboratory of Dr. James Overstreet (University of California, Davis).

Collection of Epididymal and Uterine Luminal Fluids and Sperm: Caudal epididymides were finely minced in phosphate buffered saline (PBS) at 37° C. and sperm were allowed to swim into solution for 10 min. After sperm dispersion in the suspension, tissue fragments were separated by gravity settling. The suspension was then centrifuged at 500 g for 15 min to pellet the sperm without breaking their membranes. This method, ideal for optimal cell recovery while simultaneously retaining sperm motility, is used routinely for sperm washing. The resulting fluid was further clarified by centrifugation at 16,100 g for 20 min, and the supernatant considered as ELF. Sperm pellets were washed twice by centrifugation and re-suspended in PBS.

ULF was obtained from mice artificially induced into estrous via intraperitonal injections of 7.5 i.u. equine chorionic gonadotrophin (eCG) and 7.5 i.u. human chorionic gonadotrophin (hCG) spaced 48 hr apart. Uteri were removed 13.5-14 hr later and flushed with either human tubal fluid (HTF) (Chemicon International, Temecula, Calif.), a known capacitation medium, or PBS. Protein concentrations were obtained using a biocinchoninic acid assay (BCA kit, Pierce) after the luminal fluids (2-3 ml) were subjected to centrifugation at 3,500 g for 10 min to pellet blood cells and excess tissue.

Collection of Human Spermatozoa: Freshly ejaculated sperm were collected from a fertile 25 year old male after a 3-day period of abstinence. Semen samples were allowed to liquefy at room temperature (RT) for 1 hr and then placed into 15 ml tubes and layered with Hepes-BWW medium. Samples were incubated at 32° C. for 1 hr, to allow sperm to swim out of the semen into the media. Media were collected and pooled and the sperm concentration determined using a hemocytometer.

Characterization of the Physical Nature of LFs by Ultracentrifugation: To separate the vesicular (120P) and non-vesicular (120S) fractions of ELF and ULF, samples were subjected to ultracentrifugation at 120,000 g for 2 hr, at 4° C. using a Beckman Optima L-70K ultracentrifuge and a Ti60 rotor. The supernatant, the soluble fraction, was then characterized via repeated ultracentrifugation steps at 150,000, 190,000 and 230,000 g for 8, 16 and 24 hrs respectively. All pellets were re-suspended in the initial volume of 5 ml to determine the relative concentration and form of SPAM1 in each fraction. Equal volumes of each sample were subjected to native PAGE and Western blot analysis to determine the relative amounts of SPAM1 within them. The supernatant and pellet after the final 230,000 g spin were subjected to analysis by transmission electron microscopy (TEM) as previously described.

Native Western Blot Analysis: Samples of ELF fractions collected via ultracentrifugation were subjected to Western blot analysis to detect the presence of SPAM1. Western blotting was visualized with the WesternBreeze Chemiluminescent Immunodetection Kit (Invitrogen, Carlsbad, Calif.), and all incubations carried out according to the manufacturer's instructions (at RT, with gentle shaking at ˜60 rpm). Samples of ˜20-40 μg protein from each fraction were subjected to 15% PAGE under non-reduced conditions, and transferred to a nitrocellulose membrane overnight (200 mAmps, 4° C., in transfer buffer). Bovine serum albumin (BSA, 2% W/V in PBS) was used as a negative control in one lane of the gel. The membrane was incubated in blocking solution (2% BSA), for 30 min. After decanting the blocking solution, the membrane was rinsed twice in 20 ml ddH₂O for 5 min, and incubated in 10 ml SPAM1 anti-serum (suspended in 2% BSA blocking solution, 1:1000 dilution), for 1 hr. After washing, it was incubated in 10 ml of anti-rabbit IgG secondary antibody solution provided with the kit for 30 min, washed, and rinsed twice in 20 ml ddH₂O. Signal was detected using the Chemiluminescent substrate, also provided with the kit.

Efficiency of transfer of SPAM1 from the soluble (230S) and insoluble (230P) sub-fractions of the non-vesicular LF fractions: Soluble and insoluble sub-fractions of the non-vesicular fraction of LFs were obtained by subjecting the 120S supernatant to ultracentrifugation at 230,000×g. Caudal sperm were incubated in the supernatant or the pellet, or PBS+BSA used as a control for 2 hr, at 37° C. Post-incubation, sperm were immunostained and analyzed for SPAM1 uptake via flow cytometry. Samples were incubated in SPAM1 anti-serum (1:400) for 1 hr at RT. They were washed thrice in PBS, incubated in FITC-labeled goat anti-rabbit IgG (1:400) for 30 min, in the dark at RT, and washed thrice in PBS. Samples were then analyzed using a FACSCalibur (Becton Dickinson, San Jose, Calif.) flow cytometer, which uses an argon laser at 488 nm with detectors for FITC (FL-1) and a Cell Quest software package. For each treatment, analysis of 50,000 cells was attempted. Except otherwise specified, experiments were run in triplicates.

Identification of the GPI-anchor of SPAM1 in the soluble 230S supernatant of the non-vesicular LF fraction and its role in SPAM1 transfer: Phosphatidylinositol-specific phospholipase C (PI-PLC) was added to a final concentration of 5 U/ml to remove the GPI anchor from SPAM1 and other proteins in the soluble 230S sub-fraction of the LFs. The negative control was the LFs treated with the vehicle for the enzyme. Digestion was performed at 30° C. for 15 min, after which the enzyme was inhibited with treatment of manoalide (40 μg/ml) in ethylene glycol for 20 min at 37° C. as described. Manoalide was obtained from Wako Pure Chemical industries (Mountain View, Calif.). After enzymatic inhibition, the fraction as well as its undigested vehicle control, also treated with the manoalide, was incubated with caudal sperm to allow SPAM1 transfer. The latter was detected using flow cytometry.

The effect of plasma lipoproteins containing APOJ on SPAM1 Transfer to Sperm from the 230S liquid phase of the LF/Lipoprotein Isolation and Effect of concentration on SPAM1 Transfer from ELF: Rat or mouse serum lipoproteins were isolated by density ultracentrifugation. Rat blood was subjected to centrifugation at 2000 g for 20 min to pellet RBCS. The density of the resulting rat serum was increased to 1.21 g/ml by adding 1.41 g sodium bromide (NaBr) to a final volume of 5 ml (adjusted with water to a final weight of 6.05 g) and ultracentrifuged at 230,000 g for 48 hr. The protein concentration of the resulting supernatant (shown to contain apolipoproteins via dot blot analysis for high density lipoproteins and APOJ, data not shown) was determined by our BCA kit.

ELF 230S samples were supplemented with rat lipoproteins (concentrations ranging from 40-160 μg/ml) for 20 min before incubation with caudal mouse sperm; sperm incubated in ELF+the NaBr carrier or in BSA alone were used as controls. Sperm were analyzed for SPAM1 acquisition via immunostaining followed by flow cytometry, as described above.

Lipoprotein Removal of SPAM1 from Mouse and Human Sperm and Delivery to Human Sperm: The results of the previous experiment suggested that higher apolipoprotein concentrations removed SPAM1 from the sperm surface. Thus very high concentrations of lipoproteins were used for incubating mouse (800 μg/ml) and human (8 mg/ml) sperm at 37° C. for 15-20 min. To confirm the removal of SPAM1, a fraction of the lipoprotein supernatant recovered after pelleting sperm was subjected to Western blot analysis and showed SPAM1 to be present. The remainder of the lipoprotein supernatant containing GPI-linked sperm proteins was diluted (to 8, 16, or 80 μg/ml) with PBS. These diluted samples were used for incubation of fresh populations of human sperm for the delivery of SPAM1. Following incubation for delivery of human SPAM1 to human sperm, as was the case with its removal from sperm membranes, cells were immunostained for flow cytometric analysis of SPAM1. Sperm incubated in PBS+1.21 g/ml NaBr (lipoprotein carrier) or in PBS+80 μg/ml of fresh lipoproteins served as negative controls.

The effect of Antibody Inhibition of Endogenous APOJ in 230S on SPAM1 Transfer: To determine if endogenous APOJ is involved in SPAM1 transfer from ELF and ULF 230S, APOJ was inhibited with anti-APOJ antibodies (Ab) added to the 230S subfraction 30 min prior to incubation of sperm. Thus caudal sperm were incubated at 37° C. in PBS, LF 230S+preimmune serum (PIS, 1:1000) as the controls, or LF 230S+APOJ Ab (1:1000) for 2 hr. After incubation, samples were washed twice in PBS, and stripped of the APOJ Ab from the sperm surface. This step was necessary since both the APOJ and SPAM1 antibodies are rabbit polyclonals and would bind to the same secondary Ab. Stripping of APOJ Ab with 3M acetic acid was shown to be ineffective and therefore all samples were stripped of APOJ Ab with 1 M KCl (pH 7.2) for 15 min at RT, after which they were washed 3× in PBS, immunostained for SPAM1 and subjected to flow cytometric analysis.

The Effect of low concentrations of Lipoprotein Supplements and APOJ Ab Inhibition on SPAM1 transfer from LF 230S: The effect of low concentrations (5, 10, 15 and 20 μg/ml) of rat or mouse lipoprotein on SPAM1 delivery from ULF 230S, was investigated. To determine if the enhancement of uptake was partially due to an interaction of APOJ with SPAM1, the lipoprotein supplement was APOJ antibody-inhibited prior to introduction to LF and sperm in a parallel set of samples. Thus lipoprotein samples were subjected to APOJ Ab or PIS (1:1000) for 30 min prior to their introduction to ULF 230S. Caudal sperm (5×10⁴) were incubated in each sample for 2 hr at 37° C. After incubation samples were exposed to 1 M KCl treatment to strip the APOJ Ab, followed by SPAM1 immunostaining and flow cytometry as described above.

Co-Immunoprecipitation of APOJ and SPAM1 in ULF and ELF: To determine the presence of an association between SPAM1 and APOJ in LFs, co-immunoprecipitation was performed on the ELF and ULF 230S fractions. Samples (1 ml) were treated with PIS (1:1000), APOJ Ab (1:1000) or SPAM1 Ab (1:1000), overnight at 4° C. before incubation with 125 μl Seize X Protein A beads (Pierce, Rockford, Ill.)) overnight at 37° C. Beads were washed 3× in 1×PBS and equi-volume (20 μl) samples were treated with 100 mM DTT in sample loading dye and heated to 60° C. for 5 min to extract immunoprecipitated proteins. Immunodetection of SPAM1 and APOJ (1:1000) was performed via Western analysis as described above.

Dosage Effects of Recombinant Human APOJ on SPAM1 transfer from ELF 230S: To validate the role of APOJ in SPAM1 transfer, ELF 230S samples were supplemented with recombinant human APOJ (Prospec Tany TechnoGene Ltd., Rehovot Science Park, Rehovot, Israel). This protein which is N-linked glycosylated was expressed in human cells (293 HEK) and therefore likely to have the correct glycosylation pattern. Varying concentrations of human APOJ (purity >95%), ranging from 10-100 ng/ml and solubilized in deionized water, were added to the samples before their incubation with caudal mouse sperm. Control samples received only PBS-BSA or the unsupplemented ELF 230S. Experiments were performed in duplicates. It should be noted that across a broad range of mammalian species, including humans, APOJ has a high degree (70-80%) of sequence similarity.

### Results

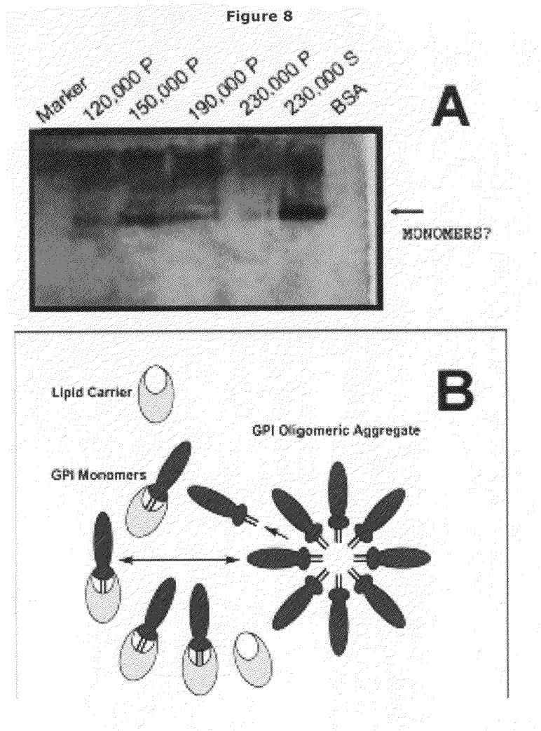

Native Western analysis showed that the BSA control gave no signal, while both low and high MW forms of SPAM1 complexes were detected in sub-fractions of the LFs with each ultracentrifugation. However the proportions varied after each spin. The low MW SPAM1-complex was most, and least, abundant in the 230,000 g supernatant (230S) and pellet (230P), respectively (FIG. 8) which are likely monomeric- and oligomeric-rich sub-fractions. TEM analysis revealed that LF 230S was entirely membrane-free (data not shown), while LF 230P contained irregularly shaped and sized micellar objects. A model proposing a dynamic equilibrium between monomers and aggregates in LFs is presented in FIG. 8.

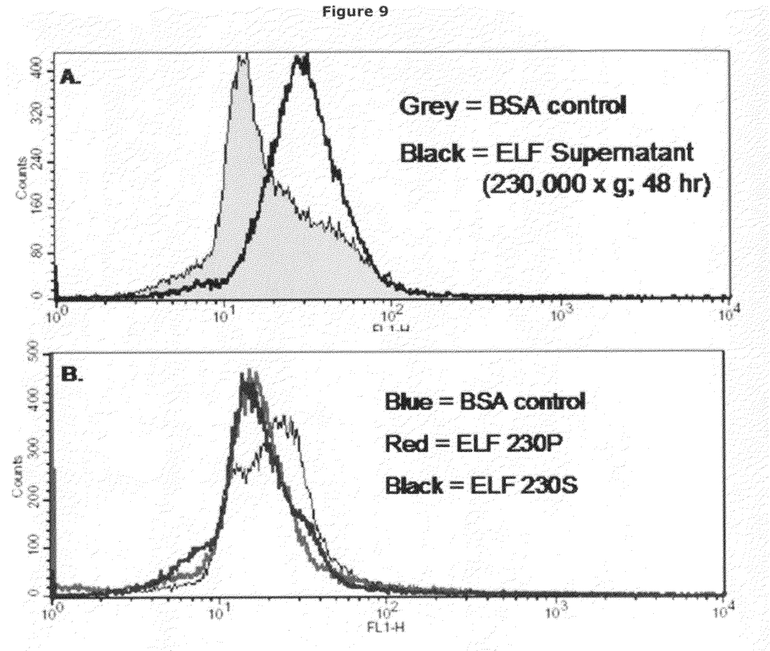

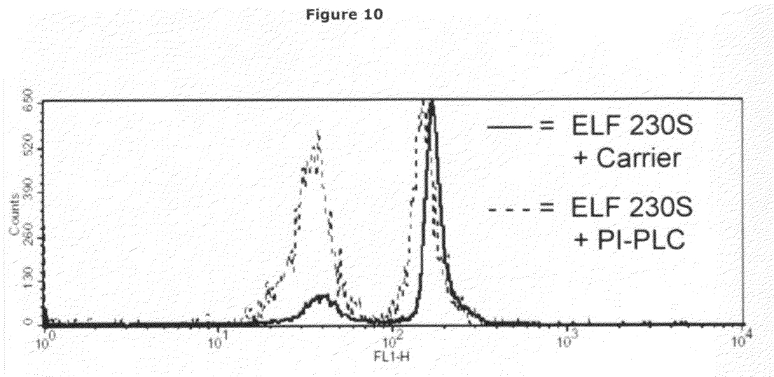

Caudal sperm incubated in ELF 230S acquired demonstrable levels of SPAM1 when compared to those incubated in BSA as seen by an increase in fluorescence intensity (FIG. 9), unlike those incubated in 230P under identical conditions (FIG. 9). This indicates that the primary form of SPAM1 transferred to the sperm surface from the soluble fraction of LFs is found in the 230S and not the 230P sub-fraction. Importantly, we demonstrated that the 230S monomeric fraction contains an intact GPI anchor which when enzymatically cleaved, dramatically reduces uptake from ELF (FIG. 10). Similar results were obtained for ULF 230S (data not shown).

When 40-160 μg/ml of rat plasma lipoproteins were added to the 230S ELF, the level of SPAM1 transfer varied with the concentration. While 40 μg/ml showed an increased uptake with respect to the carrier control, the 80 and 160 μg/ml samples showed a dose-related inhibition of SPAM1 transfer (FIG. 4). This demonstrates that lipoprotein(s) are involved in the acquisition of SPAM1 on the sperm surface in a dose-related manner.

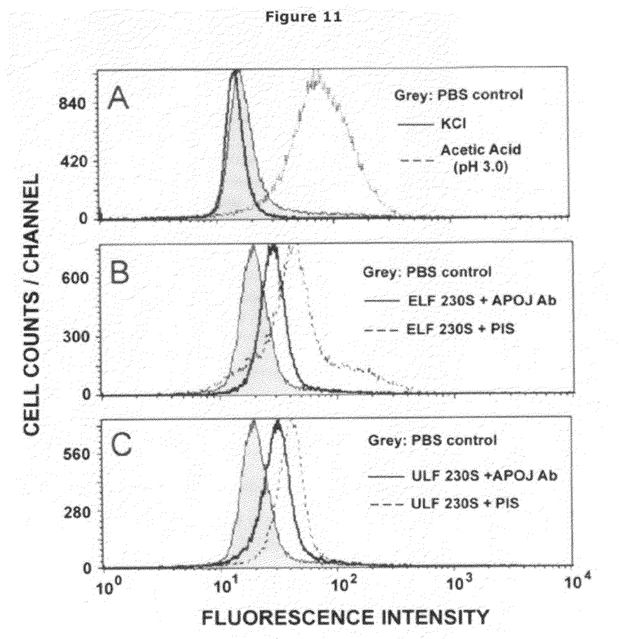

Uptake of SPAM1 was shown to be decreased by addition of APOJ Ab to both ELF and ULF 230S (FIG. 11), suggesting that APOJ plays a role in SPAM1 delivery to the sperm PM. Further support for this comes from the results of Ab inhibition of APOJ in rat serum lipoprotein supplements prior to introduction to ULF 230S (FIG. 5 v-viii). The fact that addition of APOJ Ab negated lipoprotein-mediated enhancement of SPAM1 uptake (compare FIG. 5 v-viii with i-iv) demonstrates that APOJ plays a major role in the transfer of monomeric SPAM1 from the reproductive LFs. The results show that 5-20 μg/ml of lipoproteins dose-dependently increased SPAM1 uptake from ULF by caudal sperm, with the maximal uptake seen at 10 μg/ml. Higher doses reversed this effect, explaining the inhibitory effects seen with the 80 and 160 μg/ml for the ELF sub-fraction in FIG. 4. Thus lipoprotein concentration operates bimodally with an increase in SPAM1 transfer with dosage followed by a decrease with increasing doses.

The reduced SPAM1 levels on sperm exposed to high concentrations of lipoproteins and therefore high concentrations of APOJ may result from the removal of SPAM1 already present on the sperm PM. This is supported by the finding that very high concentrations (800 and 8,000 μg/ml) of plasma lipoproteins are able to remove considerable amounts of SPAM1 from human and mouse sperm (FIG. 6A). Western analysis showed that the lipoprotein supernatant recovered after pelleting human sperm revealed the presence of the 64 kDa human SPAM1 (data not shown). Importantly, when the recovered lipoprotein supernatant with solubilized human SPAM1 was diluted with PBS to ˜80, 16 and 8 μg/ml and incubated with fresh human sperm, SPAM1 acquisition was shown to occur (FIG. 6B), indicating that SPAM1 and other GPI-linked proteins are removed from the sperm plasma membrane by lipoproteins with their lipid anchors intact. Sperm incubated in ˜16 μg/ml lipoprotein supernatant acquired the most SPAM1 when compared to those incubated in 80 and 8 μg/ml. This is consistent with the findings for mouse sperm which showed maximal enhancement of SPAM™ uptake at lipoprotein concentrations of 10-15 μg/ml (see FIG. 5).

As seen in FIG. 6B, untreated sperm have a bimodal distribution wherein one population of sperm highly expresses SPAM1. In the population of sperm expressing lower levels of SPAM1, SPAM1 receptors are unsaturated. After treatment with low levels of lipoprotein/APOJ in the presence of SPAM1, treated sperm have a single mode distribution wherein all sperm highly express SPAM1. Notably, all treated sperm express SPAM1 at physiological levels. The treatment eliminates sperm expressing lower levels of SPAM1 rather than increase the SPAM1 expression of sperm beyond physiological levels.

In FIG. 3 we show an association or interaction between SPAM1 and APOJ in both ELF and ULF, using reciprocal co-immunoprecipitation. Importantly, Western blot shows that the major 67 kDa isoform of mouse SPAM1 that is present in ELF and ULF 230S can be immunoprecipitated from these fluids by APOJ antibody, but not preimmune serum (PIS) (FIG. 3A). Similarly, when SPAM1 antibody was used for immunoprecipitation and APOJ Ab for Western, APOJ was detected (FIG. 3B). Together these findings strongly support a role for APOJ in the transport of SPAM1 monomers to the sperm surface.

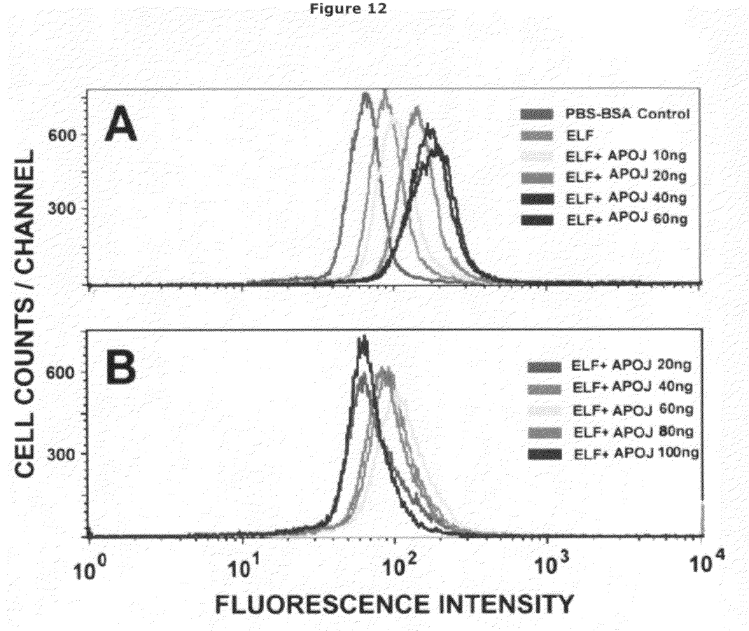

Finally, when purified recombinant human APOJ was added to ELF 230S prior to incubation with caudal mouse sperm, there was a dose-dependent effect on SPAM1 transfer. Compared to the untreated samples, uptake increased steadily in sperm incubated in the presence of recombinant APOJ at concentrations of 10 to 60 ng/ml. The latter resulted in sperm with the highest amount of SPAM1 transfer (FIG. 12). At dosages >60 ng/ml of APOJ the level of SPAM1 on sperm decreased, and at 100 ng/ml it was similar to that seen at 10 ng/ml (FIG. 12B). Effective concentrations of purified recombinant human APOJ which increase sperm uptake of SPAM1 range from about 1 to 150 ng/ml, preferably from about 40 to 80 ng/ml.

### DISCUSSION

Following the removal of vesicles from LFs, ultracentrifugation at >120,000 g and native Western analysis revealed that in the soluble membrane-free fraction SPAM1 exists in high and low MW complexes. At 230,000 g, these low and high MW complexes were predominantly present in the supernatant and the pellet, respectively. The latter was shown by TEM to consist of micellar oligomeric aggregates while the low MW component showed no structure and is likely to be monomeric. As suggested in FIG. 8, hydrophobic interactions of the GPL-anchors of SPAM1 are likely responsible for stabilizing the molecules in oligomeric aggregates.

While the insoluble aggregates were unable to deliver SPAM1 to the sperm surface, under the identical conditions the monomeric fraction was able to do so. This was also the case for insoluble vesicles which were earlier shown to transfer the protein. Since the vesicles were reported to dock on the sperm membrane at specific sites (lipid raft-associated domains) during transfer, it is likely that docking is a receptor-mediated event. Thus vesicles may carry receptors that are not present on oligomeric aggregates which are therefore not targeted to the membrane. When aggregates were resuspended in PBS and Western analysis performed on native gel there was solubilization as detected by low MW SPAM1, while SDS-PAGE revealed the 67 kDa mouse protein. We also noted that these aggregates could be solubilized in lipoproteins, suggesting that oligomeric SPAM1 may serve as a source or pool of monomers. By so doing they may regulate GPI-linked protein transfer to the membrane to effect maturation in an incremental and timely manner.

The model depicted in FIG. 8 proposes that a population of GPI-linked monomers might be stabilized and solubilized in an aqueous solution by forming hydrophobic interactions with a lipid carrier. If these carriers are targeted to the sperm surface, as is the case with APOJ, they would be ideal vehicles for transporting GPI-linked proteins to the plasma membrane where the GPI anchors could be inserted into the outer layer of the lipid bilayer via hydrophobic insertion. Importantly, the monomeric fraction of SPAM1 which was shown to have the GPI-anchor accessible to enzymatic cleavage also had the anchor in a form in which it could be inserted into the sperm plasma membrane, as evidenced by the fact that after treatment with PI-PLC SPAM1 transfer from 230S was markedly reduced. Thus the monomeric form of GPI-linked proteins, and not aggregates, is the primary form from which protein transfer occurs via the soluble membrane-free fraction of the luminal secretion.

The efficiency of SPAM1 transfer from the ELF monomeric fractions was shown to increase in the presence of rat serum lipoprotein concentrations that were 40 μg/ml or less. This is consistent with the increase in SPAM1 uptake from ULF supplemented with 5-15 μg/ml of rat serum lipoproteins (FIG. 5 i-iv), and also that from ELF supplemented with the same concentration of mouse serum lipoproteins. The identical findings with mouse and rat serum lipoproteins suggest that there were no cross-species effects with respect to the source of lipoproteins that enhanced SPAM1 uptake. When high concentrations (>40-160 μg/ml) of lipoproteins were added to ELF 230S the amount of SPAM1 on sperm was markedly reduced (FIG. 4), suggesting that high lipoprotein concentrations sequester SPAM1 from the sperm plasma membrane. This was confirmed when ˜800 μg/ml of rat serum lipoproteins was shown to dramatically remove SPAM1 from the membranes of mouse sperm (FIG. 6A). Similarly, SPAM1 could be solubilized from human sperm with high concentrations of lipoproteins, as detected via Western analysis. When the lipoproteins with solubilized human SPAM1 were diluted to 16 μg/ml and incubated with a population of fresh human sperm, SPAM1 transfer was optimal (FIG. 6B). These findings which clearly demonstrate how lipoprotein concentration is important in both the delivery and removal of SPAM1 from the sperm plasma membrane, argue for an important role of lipid carriers present in the LFs other than their role in cholesterol efflux.

APOJ has been implicated in lipid efflux from the sperm plasma membrane during capacitation. It has been shown that in the epididymal fluid it has a major fraction that is free or loosely associated with sperm, while a smaller fraction is tightly associated with the lipid bilayer. It is possible that the free fraction of APOJ is involved in stabilizing GPI-linked protein monomers while the fraction that is tightly associated with the sperm membrane is in the process of delivering GPI-linked proteins to the sperm. When APOJ was antibody-inhibited in lipoprotein supplements for the 230S fraction, enhancement of SPAM1 transfer was negated (FIG. 5 v-viii). This demonstrates its involvement in SPAM1 transfer. The finding that antibody inhibition of epididymal and uterine APOJ in the monomeric fractions also diminished transfer confirms the involvement of this lipid carrier in SPAM1 delivery to the sperm surface. Further confirmation of its role in transferring SPAM1 was obtained by the results of co-immunoprecipitation which showed an intimate association between SPAM1 and APOJ. This association is consistent with the report that APOJ forms hydrophobic complexes with the GPI-linked prion protein which is found in the soluble phase of ELF of rams. Therefore prion protein in rams may potentially be another protein delivered to the sperm surface via APOJ.

The role of APOJ in the transfer of SPAM1 to the sperm surface was validated by the use of purified recombinant protein. As was the case with rat serum lipoproteins, there was a dosage effect of the addition of purified recombinant human APOJ. The direct relationship between increasing concentration (up to 60 ng) of APOJ and SPAM1 transfer, followed by a leveling off of transfer after saturation (FIG. 12) is consistent with the formation of a complex between APOJ and SPAM1. Further, the decrease in sperm SPAM1 after sperm incubation with >60 ng/ml APOJ reflects the removal of SPAM1 from the sperm plasma membrane in the formation of a complex with the recombinant protein at high concentrations, such as above 150 ng/ml.

The finding that APOJ concentration modulates removal and delivery of SPAM1 has physiological relevance and practical implications. It has been demonstrated that the level of rat epididymal APOJ which is secreted only in the caput is ˜7-fold higher in the epididymal fluid from the caput, compared to that in the cauda. This is consistent with the fact that in the caput SPAM1 and other GPI-linked proteins are removed from the sperm plasma membrane while in the caudal epididymis they are added to the sperm. In the case of the prion protein and other GPI-linked proteins associated with the soluble phase, addition to the sperm surface occurs before or after ejaculation from the seminal plasma where the level of APOJ would be lower than that in the caput and cauda. From a practical stand point the work has the potential of leading to advances in technology for treating sperm pathology via the delivery of membrane-free GPI-linked molecules to enable sperm to effect fertilization in vitro and after intrauterine insemination.

In conclusion, our studies show that the soluble form of GPI-linked proteins, exemplified by SPAM1, is transported through an aqueous solution by APOJ. Implicated in cholesterol efflux from the sperm surface during capacitation as well as in other functions, APOJ has now been shown to play a definitive role in the delivery of GPI-linked proteins to sperm. To date there has been no clear evidence for a definitive role. Another apolipoprotein that could perform the same function as APOJ is APOA-1 which is also expressed in both the male and the female tract and is also implicated in cholesterol efflux. Notably it shares with APOJ the same receptor and co-receptor, megalin/cubulin, on the epithelial membrane of the epididymal and uterine tract where it mediates endocytotic removal of lipidated proteins.

Thus we put forward an expansion of the lipid-poor apolipoprotein model proposed to carry out cholesterol efflux on the plasma membrane of somatic cells and sperm, as a model for lipid exchange involving GPI anchors. In FIG. 2 we propose that APOJ (and possibly APOA-1) solubilize and stabilize GPI-linked monomers from the epithelial membranes, transport and donate them to the sperm surface where they accept cholesterol. The lipidated proteins are then endocytosed via a receptor-mediated mechanism at the epithelial cell lining. Therefore the findings of this study present a more efficient interaction of APOJ and the sperm membrane and its role in membrane remodeling than previously envisaged, while revealing the mechanism by which soluble GPI-linked proteins are delivered to the sperm surface.

The entire disclosures of all applications, patents and publications, cited above and below are hereby incorporated by reference.

What is claimed:

1. A composition comprising:
a bioactive molecule, and
a lipid carrier,
wherein at least one of said bioactive molecule or lipid carrier is substantially purified, and wherein the composition comprises about 1-150 ng/ml of said lipid carrier.

2. The composition of claim 1 wherein the bloactive molecule is selected from the group consisting of SPAM1, CD59, CD55, CDw52 and P34H.

3. The composition of claim 1 wherein the lipid carrier is selected from the group consisting of Clusterin, APOJ, Clusterin/APOJ, ApoA-1, SGP2, TRPM, gp80 and SP-40.

4. The composition of claim 1 wherein the bioactive molecule is SPAM1 and the lipid carrier is APOJ.

5. A method of enhancing fertilization comprising administering the composition of claim 1 to an animal whereby the bioactive molecule is transferred from the composition to the surface of a sperm cell in the animal.

6. A method for enhancing sperm maturation and function comprising, in an in vitro environment:
obtaining sperm from a male candidate; and
adding the sperm in vitro to a medium wherein the medium comprises a bioactive molecule and about 1-150 ng/ml of a lipid carrier.

7. The method of claim 6, wherein the bioactive molecule is selected from the group consisting of SPAM1, CD59, CD55, CDw52 and P34H.

8. The method of claim 6, wherein the lipid carrier is selected from Clusterin, APOJ, Clusterin/APOJ, ApoA-1, SGP2, TRPM, gp80 and SP-40.

9. The method of claim 6 wherein the bioactive molecule is SPAM1 and the lipid carrier is APOJ.

10. The method of claim 6 further comprising delivering said sperm to a uterine tract.

11. The method of claim 6 further comprising adding at least one egg to the medium and incubating until said egg is fertilized.

12. The method of claim 11 further comprising cultivating said fertilized egg into an embryo.

13. The method of claim 12 further comprising delivering said embryo to a uterine tract.

14. A method for enhancing sperm maturation and function comprising, in an in vitro environment:
obtaining a first sample of sperm from a first male candidate;
treating said first sample of sperm with a solution comprising lipoproteins, wherein the solution comprises an effective amount of lipoproteins to remove at least one bioactive molecule from said first sample of sperm;
recovering a supernatant from the treated solution, wherein said supernatant comprises said at least one bioactive molecule and said lipoproteins; and
adding said recovered supernatant to a second sample of sperm, whereby said at least one bioactive molecule is transferred to the surface of a sperm.

15. The method of claim 14 wherein the effective amount of lipoproteins comprises a lipoprotein concentration of at least about 40 ug/ml.

16. The method of claim 14 wherein the lipoproteins comprise APOJ.

17. The method of claim 16 wherein the effective amount of APOJ comprises at least about 150 ng/ml.

18. The method of claim 14 wherein the bioactive molecule is SPAM1.

19. The method of claim 14 wherein the second sample of sperm is from the first male candidate.

20. The method of claim 14 wherein the second sample of sperm is from a second male candidate.
